(19) United States
(12) Patent Application Publication
(10) Pub. No.: US 2022/0370805 A1
(43) Pub. Date: Nov. 24, 2022

(54) ELECTROENCEPHALOGRAPHY (EEG) ELECTRODE ARRAYS AND RELATED METHODS OF USE

(71) Applicants: Duke University, Durham, NC (US); New York University, (US); U.S. GOVENMENT AS REPRESENTED BY THE DEPARTMENT OF VETERANS AFFAIRS, Washington, DC (US); University of Utah Research Foundation, Salt Lake City, UT (US); BLACKROCK MICROSYSTEMS, LLC dba, Salt Lake City, UT (US)

(72) Inventors: Gregory Cogan, Durham, NC (US); Jonathan Viventi, Durham, NC (US); Nandan Lad, Durham, NC (US); Bijan Pesaran, New York, NY (US); Virginia Woods, Durham, NC (US); Chia-Han Chiang, Durham, NC (US); Charles Wang, Durham, NC (US); Katrina Barth, Durham, NC (US); Werner Doyle, New York, NY (US); Patricia Dugan, New York, NY (US); Orrin Devinsky, New York, NY (US); Sasha Devore, New York, NY (US); Daniel Friedman, New York, NY (US); Amy Orsborn, New York, NY (US); Florian Solzbacher, Salt Lake City, UT (US); Robert Franklin, Salt Lake City, UT (US); Sandeep Negi, Salt Lake City, UT (US); Saket Mulge, Salt Lake City (US)

(21) Appl. No.: 17/761,369

(22) PCT Filed: Sep. 18, 2020
(86) PCT No.: PCT/US2020/051400

§ 371 Date: Mar. 17, 2022

Related U.S. Application Data
(60) Provisional application No. 62/902,145, filed on Sep. 18, 2019.

(51) Int. Cl. A61N 1/36

(52) U.S. Cl. CPC A61N 1/36064

(57) ABSTRACT

The present disclosure provides systems and methods related to electroencephalography (EEG) electrode arrays. In particular, the present disclosure provides systems and methods relating to the manufacture and use of high-resolution electrocorticography (ECOG) electrode arrays and stereoelectroencephalography (SEEG) electrode arrays having various combinations and arrangements of microelectrodes and macroelectrodes for recording and modulating nervous system activity.

### RELATED APPLICATIONS

This application claims priority to and the benefit of U.S. Provisional Patent Application No. 62/902,145 filed Sep. 18, 2019, which is incorporated herein by reference in its entirety for all purposes.

### GOVERNMENT FUNDING

This invention was made with Government support under Federal Grant No. 5U01NS099697 awarded by the National Institutes of Health. The Federal Government has certain rights to the invention.

### FIELD

The present disclosure provides systems and methods related to electroencephalography (EEG) electrode arrays. In particular, the present disclosure provides systems and methods relating to the manufacture and use of high-resolution electrocorticography (ECOG) electrode arrays and stereoelectroencephalography (SEEG) electrode arrays having various combinations and arrangements of microelectrodes and macroelectrodes for recording and modulating nervous system activity.

### BACKGROUND

Epilepsy affects 3.4 million people in the US, and more than 30% of these people are unable to adequately control their seizures using medication. For patients with medically refractory epilepsy, surgical therapies such as resection, ablation and neurostimulation are useful adjuncts. Unfortunately, seizure freedom is only achieved in ˜50% of these surgical patients. In order to apply current and future surgical modalities more effectively, a more precise understanding of patient-specific seizure networks in patients' native environments is needed.

Invasive electrographic studies are the gold standard method for delineating these patient-specific seizure networks; however, these studies currently rely on low-density electrode arrays with wires tunneled through the scalp that are hard-wired to remote recording systems. Due to the risk of infection from these wires through the scalp, the electrode arrays necessitate relatively brief recording periods of one to two weeks in an epilepsy monitoring unit. Given that patient-specific seizure networks exhibit monthly variations, including even switching the hemisphere of seizure onset, and that invasive electrophysiological studies are almost always performed in an unnatural state (e.g., post-surgical implantation, receiving analgesics, undergoing rapid removal of seizure medications, and the like), neurologists and surgeons currently plan treatments using sparse and poorly representative physiologic data that does not accurately reflect a patient's typical epileptic activity. All of these factors contribute to poor surgical outcomes due to an insufficient understanding of patient-specific seizure networks.

Patients undergoing evaluation for surgery for pharmacologically-resistant epilepsy are implanted with arrays of invasive electrocorticographic (ECOG) electrodes to record seizure activity. These arrays typically contain ˜100 electrodes with 10 mm spacing. These invasive recordings offer improved signal localization and quality, but still do not capture the full spatial resolution of human brain activity.

Another major limitation for current monitoring strategies is that patients are monitored after epilepsy has developed. For patients, the sudden and unforeseen onset of seizures represents one of the most disabling acts of the disease. Being able to predict seizures would therefore offer a significant boost in therapeutic options for treating epilepsy. While past techniques for prediction were restricted to linear models using small amounts of data, more recent attempts utilized multi-day EEG recordings which offer better training and testing opportunities for machine learning based techniques. These approaches however are limited in that they are not mechanistic and cannot adequately model spatial and temporal dynamics, and so do not have the resolution to resolve seizure networks.

The use of high density recordings over long periods of time would therefore offer a unique opportunity to capture mechanistically-informed data to allow for better seizure prediction algorithms which would expand clinical therapeutic options. Higher density electrode arrays are needed to capture rich brain signals that reflect tissue excitability such as action potentials, which have been observed with microelectrodes on the surface of the brain.

### SUMMARY

Embodiments of the present disclosure include an electroencephalography (EEG) device comprising an electrode array having a plurality of microelectrodes and macroelectrodes uniformly or non-uniformly arranged in a polymer-based substrate. In accordance with these embodiments, the EEG device also includes a signal acquisition component coupled to the electrode array configured to collect and transmit electrical signals obtained from a subject's brain.

In some embodiments, the electrode array is configured to perform electrocorticography (ECoG) on the subject. In some embodiments, the electrode array is configured to perform stereoelectroencephalography (SEEG) on the subject.

In some embodiments, at least a portion of the electrode array or the signal acquisition component comprise silicone molding. In some embodiments, at least a portion of the electrode array is coated in silicone having a thickness from 0.2 mm to 2.0 mm.

In some embodiments, the polymer-based substrate comprises a liquid crystal polymer (LCP).

In some embodiments, the device further comprises a circuit coupled to the electrode array and configured to amplify and/or digitize the electrical signals.

In some embodiments, the signal acquisition component is coupled to a clinical data acquisition system.

In some embodiments, the signal acquisition component comprises at least one coiled lead comprising a plurality of microwires extending from contacts points on the plurality of microelectrodes and macroelectrodes.

In some embodiments, the at least one coiled lead comprises a single lead comprising the plurality of macroelectrodes.

In some embodiments, the at least one coiled lead comprises from 2 to 20 leads comprising the plurality of microelectrodes.

In some embodiments, the at least one coiled lead comprises from 10 to 500 channels per lead.

In some embodiments, the at least one coiled lead comprises a diameter of at least 1.0 mm.

In some embodiments, the signal acquisition component comprises a wireless chip. In some embodiments, the wireless chip is contained within a polymer-based substrate. In some embodiments, the wireless chip is configured for implantation in the subject.

In some embodiments, the wireless chip is a 65,000 electrode wireless recording chip.

In some embodiments, the electrode array comprises from 4 to 500 uniformly or non-uniformly arranged macroelectrodes. In some embodiments, the macroelectrodes comprise diameters from 1 mm to 10 mm. In some embodiments, the macroelectrodes are spaced from 0.2 cm to 5 cm apart from each other. In some embodiments, the electrode array comprises from 100 to 10,000 uniformly or non-uniformly arranged microelectrodes.

In some embodiments, the microelectrodes comprise diameters from 1 μm to 1 mm. In some embodiments, the microelectrodes are spaced from 100 μm to 5 mm apart from each other.

In some embodiments, the electrode array provides at least a 2-fold increase in spatial sampling resolution compared to an array comprising only macroelectrodes.

Embodiments of the present disclosure also include an electrocorticography (ECoG)-stereoelectroencephalography (SEEG) combination device comprising an ECoG component having an electrode array comprising a plurality of microelectrodes and macroelectrodes uniformly or non-uniformly arranged in a polymer-based substrate, and a signal acquisition component coupled to the electrode array configured to collect and transmit electrical signals obtained from a subject's brain; and an SEEG component having an electrode array comprising a plurality of microelectrodes and macroelectrodes uniformly or non-uniformly arranged in a polymer-based substrate, and a signal acquisition component coupled to the electrode array configured to collect and transmit electrical signals obtained from a subject's brain.

Embodiments of the present disclosure also include a method of manufacturing any of the EEG devices described herein. In accordance with these embodiments, the method includes arranging the plurality of microelectrodes and macroelectrodes uniformly or non-uniformly within a polymer-based substrate to form the electrode array, and coating at least a portion of the array and the signal acquisition component with a composition comprising silicone.

Embodiments of the present disclosure also include an electroencephalography (EEG) system comprising an electrode array having a plurality of microelectrodes and macroelectrodes uniformly or non-uniformly arranged in a polymer-based substrate, a signal acquisition component coupled to the electrode array configured to collect and transmit electrical signals obtained from a subject's brain, and a clinical data acquisition system.

In some embodiments, the electrode array is configured to perform electrocorticography (ECoG) on the subject. In some embodiments, the electrode array is configured to perform stereoelectroencephalography (SEEG) on the subject.

In some embodiments, the system comprises an electrode array configured to perform electrocorticography (ECoG) on the subject, and an electrode array configured to perform stereoelectroencephalography (SEEG) on the subject.

In some embodiments, the device further comprises a circuit coupled to the electrode array and configured to amplify and/or digitize the electrical signals.

In some embodiments, the signal acquisition component comprises at least one coiled lead comprising a plurality of microwires extending from contacts points on the plurality of microelectrodes and macroelectrodes.

In some embodiments, the signal acquisition component comprises a wireless chip.

Embodiments of the present disclosure also include a method of evaluating a subject for a neurological impairment. In accordance with these embodiments, the method includes recording electrical signals in a portion of a subject's brain using the any of the devices described herein, and evaluating the subject based on the recorded electrical signals.

In some embodiments, the neurological impairment comprises a seizure.

In some embodiments, evaluating the subject comprises determining the presence or absence of the neurological impairment, and/or determining the severity of the neurological impairment. In some embodiments, evaluating the subject comprises identifying means for treating the neurological impairment.

In some embodiments, the method further comprises treating the neurological impairment in the subject, wherein the treatment comprises pharmacological intervention, surgical intervention, and/or electrophysiological intervention.

Embodiments of the present disclosure also include a method for treating a subject having or suspected of having a neurological impairment. In accordance with these embodiments, the method includes recording electrical signals in a portion of the subject's brain using an electrode array comprising: a plurality of microelectrodes and macroelectrodes uniformly or non-uniformly arranged in a polymer-based substrate, and a signal acquisition component comprising a wireless chip coupled to the electrode array configured to collect and transmit electrical signals obtained from a subject's brain; and administering an electrical pulse to at least a portion of the subject's brain to treat the neurological impairment.

### BRIEF DESCRIPTION OF THE DRAWINGS

FIGS. 1A-1B: FIG. 1A includes a representative high-density hybrid electrode with 2.3 mm diameter macroelectrodes spaced 10 mm apart, and 200 μm microelectrodes spaced 2 mm apart; includes integrated ASIC for neural recording and stimulation. The 4×4 design illustrated includes 320 electrodes. An implanted wireless integrated circuit provides neural recording and stimulation. A wireless relay station outside of the body provides wireless power delivery and data transmission with the implanted array (FIG. 1A). FIG. 1B includes a representative brain MRI image showing an SEEG electrode (highlighted in red) and a high-resolution SEEG electrode with 16 macroelectrodes and 98 microelectrodes (114 total). Most contacts in conventional SEEG either straddle gray and white matter, or gray matter from different gyri producing low quality signals that are not spatially resolved, leading to poor surgical outcomes. The novel architecture of combined microelectrodes and macroelectrodes insures that multiple electrodes sample from each gray matter area along the trajectory and that an individual electrode does not erroneously sample from disparate cortical gyri (FIG. 1B).

FIGS. 2A-2C: Representative images of wired LCP μECoG arrays for intraoperative recording. FIG. 2A includes a 1,024-ch array with 80×40 mm coverage and 1.7 mm spacing.

FIG. 2B includes a 244-ch array with 12×12 mm coverage and 750 μm spacing. FIG. 2C includes a 128-ch array with 11×21 mm coverage and 1.33 mm spacing.

FIGS. 3A-3H: FIGS. 3A-3B include representative images of fabricated high-density LCP hybrid ECoG arrays designed to replace existing 4×4 cm ECoG arrays. The example shown includes 16 macro and 304 microelectrodes with 2 mm pitch. FIGS. 3C-3D include representative images of fabricated LCP ECoG strip electrodes with LCP pig tail connectors for conventional clinical macro ECoG. FIGS. 3E-3H include representative images of high-density wired interconnect systems for use in the epilepsy monitoring unit (EMU).

FIGS. 4A-4C: Representative images of an active, high-density ECoG (FIG. 4A) and SEEG (FIG. 4B) electrode arrays are shown. FIG. 4C includes a representative image of the LCP-based construction that integrates the ASIC (see FIG. 5) into both electrode array designs.

FIGS. 5A-5F: Representative images of a BRM 128-channel recording chip (Intan design) (FIG. 5A), a BRM 512-channel stimulating/recording IC (BRM design) (FIG. 5B), and a Cereplex E headstage as used in human subject studies (FIG. 5C). These exemplary wireless chips are integrated in LCP construction (see FIG. 4). FIG. 5D is a representative image of a 65,536-ch wireless recording IC in a thin, flexible form factor. FIG. 5E is a representative imager of an LCP electrode array with an embedded IC. FIG. 5F is a representative cross section of an LCP device showing the IC embedded between layers of LCP.

FIGS. 6A-6D: Representative results of interictal events with high spatial resolution recorded intraoperatively with LCP electrodes of the present disclosure. Raw data from a 244-ch LCP ECoG with 200 μm electrodes/750 μm spacing shows heterogeneous activity within the 12×12 mm² recording area (FIG. 6A). Focusing on the same interictal activity from just three electrodes on the array illustrates the spatial propagation of a single inter-ictal discharge (labeled with asterisks at left and color-coded circles at right) on a spatial map of the array (FIG. 6B). Example micro-seizure confined to just two microcontacts and not observed on neighboring electrodes (FIG. 6C). Example HFO occurring on only a single electrode and not observed on neighboring electrodes (FIG. 6D).

FIG. 7 includes representative results of HFO size distribution. Approximately 88% of detected HFO events only occurred on a single channel at a time and were not detected on neighboring microelectrode channels 1.7 mm away.

FIG. 8 includes representative results of thin film (TF) LCP electrode lead resistance before and after 47,000 bend cycles. All 122 channels remained functional with no increase in resistance.

FIGS. 9A-9B: μECoG arrays capture neural activity in the high-gamma frequency band at significantly higher power than macro-ECoG or SEEG during (FIG. 9A) auditory speech perception and (FIG. 9B) motor speech production. ***p<0.001, Mann-Whitney U test. Red lines from top to bottom indicate 25, 50, and 75 percentile of high-gamma distribution. Number of electrodes for speech perception: SEEG—265, macro—68, μECoG—133. Number of electrodes for speech production: SEEG—198, macro—17, μECoG—115.

FIG. 10: Bending force (mean±std) of the thin film (TF) LCP-based electrode arrays and a clinical grid (CG) electrode array by Ad-Tech. The TF LCP-based electrode array device is thinner than the Ad-Tech electrode (200 μm vs. 850 μm), resulting in greater flexibility and smaller bending force.

FIGS. 11A-11B: Kriging analysis in broad-band human motor cortex. FIG. 11A shows variance of signal differences (semivariance) as a function of electrode spacing (black squares) was fit with a Matérn covariance function (red line) parameterized by range (theta) and smoothness (nu). Interpolation from 1.5 mm spacing (twice the 750 μm array spacing) had low error for this long range and smooth field. FIG. 11B shows a shorter range, rough field was interpolated with larger error. The spacing required to normalize interpolation error to 10% would be 1.24 mm.

FIG. 12: Kriging analysis during speech audition. A five-phoneme non-word was played at time zero while μECoG was recorded from auditory cortex. Modulation of average HG power was seen from ˜200-2000 ms following speech. The spatial variation also increased in this window as seen by the larger standard deviation interval. Spatial covariance models were fit on sequential latent states in the power envelope signal. The kriging resolution for ideal, noiseless models varied from several millimeters to 200 μm, depending on spatial complexity. Accounting for recording noise, kriging resolution was 80-1000 μm.

FIG. 13: Representative image of a μECoG/μSEEG combination device (e.g., flex EL244 with hybrid SEEG electrode), according to one embodiment of the present disclosure.

FIG. 14: Representative widefield microscope images of the cortical surface obtained following implantation of “flexible” (left panel) and “stiff” (right panel) molded electrode arrays, according to one embodiment of the present disclosure. The electrode arrays differ in the thickness of the molded silicon (stiff at 1 mm; flexible at 0.2 mm). Data reporting brain deformation, the rate of edema measured due to the associated inflammatory response, and whether neurological signs were observed during implantation were obtained. Note the increased redness, increased vessel thickness due to engorgement with blood, and overall increased vascular inflammatory response following implantation of the stiff array compared with the flexible array.

FIG. 15: Representative image and schematic (inset) of the heat press assembly used to thermally-fuse layers of LCP to form the electronics package.

FIG. 16: Representative recording chamber system designed to test wired and wireless SEEG electrodes in nonhuman primates.

FIG. 17: Representative diagrams of a coiled lead design for an electrode array, according to one embodiment of the present disclosure (left panel). Micro wires from the array contacts are fully encased in the liquid crystal polymer (LCP) substrate and extend from the array in the form of flat, flexible strips. These flat strips are coiled and adhered within a silicone/polyurethane structure to form the leads. Representative method steps for coiled lead formation according to one embodiment of the present disclosure (right panel).

FIG. 18: Representative diagrams of a connector design for an electrode array, according to one embodiment of the present disclosure. The end of the LCP wiring strip contains a series of micro-contacts to connect with a compression board.

### DETAILED DESCRIPTION

The present disclosure provides systems and methods related to electroencephalography (EEG) electrode arrays. In particular, the present disclosure provides systems and methods relating to the manufacture and use of high-resolution electrocorticography (ECOG) electrode arrays and stereoelectroencephalography (SEEG) electrode arrays having various combinations and arrangements of microelectrodes and macroelectrodes for recording and modulating nervous system activity.

Utilizing microscale electrode arrays for the treatment and prevention of neurological diseases and disorders, such as seizures, offers higher resolution, improved detection rates, and can potentially offer precursors that predict clinical events. For example, microscale electrode arrays can better detect high-frequency oscillations and microseizures, which are either not observed on existing macroelectrodes or poorly sampled. These are known markers of the seizure onset zone but are not yet used for diagnosis.

The use of these “microsignals” to better inform seizure diagnostics, and more broadly, in research on cognition and brain-computer interfaces, motivated several electrode manufacturers to integrate a selection of microelectrodes into clinical macroelectrode grids. Several have received FDA approval, but several technical challenges remain that limit their widespread adoption. First, commercially available microwire grids have very high and varying impedances (6-8 MΩ to <100 kΩ) owing to their non-uniform exposure from the insulating substrate. They also have “sharp, ragged edges” along the metal sensing surface that has caused damage to the brain and prompted a recall. Finally, interfacing microelectrodes with existing clinical data acquisition (DAQ) systems leads to notable signal distortions because microelectrodes have much higher impedances than macroelectrodes (<5 kΩ) and require amplifiers with higher input impedances to produce reliable, low noise and independent recordings. These shortcomings highlight the need for an improved, high-resolution diagnostic tool to better study, treat, and prevent neurological diseases and disorders, such as epilepsy.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, neurobiology, microbiology, genetics, electrical stimulation, neural stimulation, neural modulation, and neural prosthesis described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. ELECTROCORTICOGRAPHY (ECoG) AND STEREOELECTROENCEPHALOGRAPHY (SEEG) ELECTRODE ARRAYS

Embodiments of the present disclosure include the manufacture and use of high-density, high-channel-count liquid crystal polymer (LCP) smart electrocorticography (ECoG) and stereoelectroencephalography (SEEG) electrode arrays for use in the clinical standard of care for monitoring and treatment of refractory epilepsy. In some embodiments, the ECOG and SEEG electrode arrays include >300 channel passive LCP macro/micro-electrocorticographic (μECoG) electrodes and >100 channel macro/micro-stereoelectroencephalographic (μSEEG) electrodes, covering both electrode types with one technology. In accordance with these embodiments, chronic, in vivo demonstrations of up to 1024 channel active, smart electrodes can be used to generate and make available all necessary data (e.g., for FDA Investigative Device Exemption IDE applications) and immediate chronic in vivo use as research market tool. The electrode arrays of the present disclosure can also serve as platform for growing numbers of clinical and basic neuroscience research leveraging epilepsy patient surgical procedures to place experimental devices for acute or chronic neural recording to advance understanding and treatment of other neurological disorders (e.g., speech/communication/locked in syndrome, depression, etc.). As described further herein, the ECoG and SEEG electrode arrays of the present disclosure demonstrated efficacy at lower channel counts in animal and human subjects and have led to improved clinical outcomes, safety, and patient comfort compared to conventional ECoG and SEEG electrodes. ECoG and SEEG electrode arrays are compatible with the current standard of care and clinical workflows, and significantly improve patient comfort, risk profile, and clinical workflow, leading to better outcomes and a paradigm change in Epilepsy surgery.

Embedded electronics using existing application specific integrated circuits (ASICs) have been used to amplify, digitize, multiplex, and process up to 1024 electrode channels, allowing monitoring of electrode health (e.g., impedance spectra), identify electrodes damaged/trimmed during surgical implantation, and provide automatic mapping with suitable clinical monitoring tools. This significantly reduces errors in mapping and time to connect electrodes, making high-channel-count neural monitoring feasible in clinical practice. Within HIPAA constraints, the electrode arrays can store limited patient information, e.g., for traceability.

The ECoG and SEEG electrode arrays of the present disclosure comprise a hybrid design that includes both macro- and microelectrodes in the same device, which facilitates the integration of clinical practice with advances in research that have so far not been clinically useful or accepted due to lack of clear correlation between microelectrode and video EEG/ECoG/SEEG data, and a paucity of demonstrated improved outcomes in human subjects. This lack of demonstrated clinical utility has led to continued use of decades old technology and rejection of advanced electrodes in clinical epilepsy monitoring.

In some embodiments, the LCP active electrodes embed electronics that amplify, digitize, and multiplex more than 1000 electrode channels. They monitor electrode health (e.g., impedance spectra), identify electrodes damaged/trimmed during surgery and provide automatic mapping with suitable clinical monitoring tools. In some embodiments, each active electrode array uses a single 8 wire pigtail with a high-bandwidth digital interface, regardless of channel count. The low power interface allows the number of electrodes on each array to scale to thousands of channels, without increasing the wiring burden or infection risk for the patient. For example, one embodiment of the Application Specific Integrated Circuit ASIC includes: <0.3 mW/5×5 mm² per 32 channel tile, <10 mW/1000 channels (i.e. <40-80 mW/cm² safety limit), independent recording/stimulation control on every channel, adjustable bandwidth from 1 Hz to 10 kHz, gain up to 200, stimulation currents of 0.5 μA to 3.8 mA, 0.125 Hz-2 kHz, 0.1 ms-1 s, with stimulation monitoring of 1:11 on electrode voltage, 30 kSps, 16 bit ADC, 12 bit ENOB. As described further herein, LCP ECoG and SEEG electrodes with macro/micro configurations and up to 300 channels (passive) and 1024 channels (active) were designed, built, and tested.

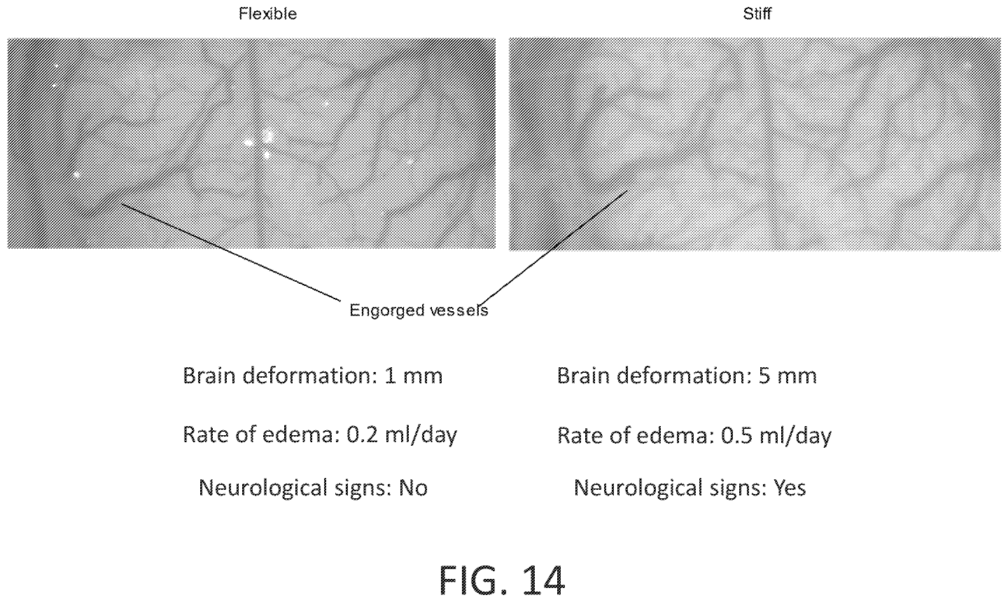

The ECoG and SEEG electrode arrays of the present disclosure address long standing clinical needs not met by current technology. For example, the ECoG and SEEG electrode arrays of the present disclosure reduce errors in mapping due to erroneous connections of conventional leads with touchproof connectors for each channel, and reduce the time to connect electrodes, making high channel neural monitoring feasible in clinical practice. In some embodiments, the electrodes include ultra-thin (e.g., 20 μm) LCP as substrate and encapsulation with Pt/IrOx contact metallization. LCP allows embedding of bare Si ASICs and passive components into the LCP substrate, creating similar functionality as monolithic Si active electrodes (e.g., Neuropixel), but with the ability to integrate suitable electronics with suitable electrode architectures (hybrid integration), without the need to redevelop the entire device to accommodate changes in architecture. Biocompatibility, ion and water barrier properties of the LCP are superior to polymer based thin film materials in vivo. The high Young's modulus of LCP with high bendability (thin material) combines stiffness in longitudinal direction (e.g., to push a strip under the Dura or insert an SEEG electrode) with low forces on the brain in transverse direction, improving patient comfort and complication rates (see, e.g., FIG. 14).

The ECoG and SEEG electrode arrays of the present disclosure incorporate the concept of using thin film technology on polymer substrates for thinner and more flexible electrode arrays. Polymer materials as well as very thin semiconductor-based high channel electrodes have been proposed, but none have become an accepted clinical product (e.g., for Epilepsy monitoring). The ECoG and SEED electrode arrays described herein address clearly defined clinical needs and usability requirements that lead to products for clinical use, including enabling more advanced higher resolution, and high channel and previously undocumented self-testing and mapping capabilities. The ECoG and SEEG electrode arrays of the present disclosure provide many advantages over current electrode arrays, including but not limited to, the integration of LCP technology that has low water uptake, allowing the use without additional encapsulation; the ability to be processed like printed circuit board material for direct assembly and embedding of bare silicon or chip-scale packaged electronics without an additional interposer or substrate; self-test, automatic channel mapping and high channel count enabled through hybrid embedded electronics; improved material and fabrication processing that enables the above with line widths and resolution that allow flexible electrode architectures; high Young's modulus for mechanical strength in insertion/lateral pushing of electrodes combined with low profile designs yields high bendability and low forces on neural tissue to improve patient comfort; manufacturing costs that are at least 2× lower than conventional ECoG and SEEG electrodes and at least 10× lower than most cleanroom thin-film fabrication processes per cm² of surface area; and demonstrated data in vivo (animal/human) and in vitro indicating>5 year lifetime.

Reproducible and robust LCP-based ECoG and SEEG electrode arrays were developed. Mechanical testing devices comprising 100 functional passive electrodes were first generated. Layer thicknesses were characterized on monitor wafers using an FEI Nova 200 focused-ion beam (FIB) imaging tool. Inspections for visual damage were performed using SEM, soak in PBS buffer solution at 67° C. (or 77° C. if LCP ageing models permit), and electrical leakage currents and impedance spectroscopy (Gamry 600) were determined for electrodes without exposed electrode sites. Electrodes were inspected using scanning electron microscopy (SEM) for complete LCP removal over the contacts. New masking foils were used for the deposition of sputtered PtIr. A titanium/iridium oxide bilayer coating was deposited onto the electrodes by reactive sputtering in a TMV multi cathode DC magnetron sputtering system. The Ti adhesion layer was deposited from Ti targets using Ar plasma at 100 W and at 4 mTorr for 4 minutes. The iridium oxide layer was deposited from iridium targets using a mixture of Ar, O₂ and H₂O gases at 100 W and at 30 mTorr for 25 minutes. Target film thicknesses (20 nm for Ti and 270 nm for IrOx) were measured to be on flat silicon control samples that are cleaved and SEM imaged in cross-section view.

A Zeiss Supra 40 SEM was used for inspection of the electrodes. A typical acceleration voltage of 1 kV was used in order to avoid charging effects and the need of coating the specimen with a discharge layer. A Zeiss Stereo light microscope was used to inspect LCP uniformity, array packaging quality and large-scale features of the arrays. Periodic trace material analysis of PBS solutions for Ir and Pt dissolution using inductively coupled plasma mass spectroscopy (ICPMS) confirmed the lack of contamination.

LCP encapsulation stability was tested using saline soaking at elevated temperature (e.g., 67° C. or 77° C.). Impedances remained within original tolerances of current LCP product devices (<1 kΩ (macro)<10 kΩ (micro contacts) at 1 kHz) and minimal LCP degradation was observed (using optical and electron microscopy) for initially >2 months, then ≥3 years of equivalent lifetime. Test sample sizes were based on Weibull and Binomial Distribution. Projected sample numbers were 23 devices at 0 failures and 90% reliability/90% confidence factor and 39 devices at 1 failure.

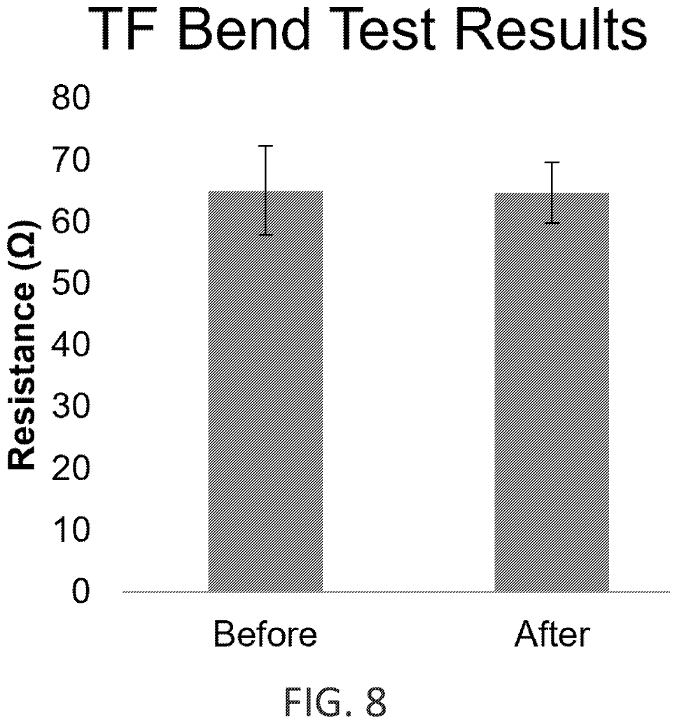

To better understand changes in electrode performance and identify possible failure mechanisms in vivo, electrochemical measurements (CV, EIS) were done both before implantation and throughout the indwelling period. Prior to implantation, all electrodes were measured in model interstitial fluid (ISF) in a 3-electrode configuration with Pt counter and Ag|AgCl reference electrodes. CV measurements were done from −0.6 to 0.8 V versus the Ag|AgCl reference. This potential region was identified as the water window for PtIr contacts. CV data were collected using 50 mV/s and 50 V/s sweep rates with 10 mV step size. EIS measurements were done using 10 mV RMS about the open circuit potential from 10⁵ to 10° Hz with 10 points per decade. All electrochemical measurements were carried out using a Gamry 600+ potentiostat (Gamry Instruments, USA) and a Gamry ECM8 Electrochemical Multiplexer. LCP encapsulation on electrodes met mechanical, electrical and chemical (dissolution) requirements under accelerated ageing testing (>2 months) with 90% reliability and 90% confidence factor (see, e.g., FIG. 8).

In accordance with the above, embodiments of the present disclosure include an electroencephalography (EEG) device comprising an electrode array having a plurality of microelectrodes and macroelectrodes uniformly or non-uniformly arranged in a polymer-based substrate. The EEG device also includes a signal acquisition component coupled to the electrode array configured to collect and transmit electrical signals obtained from a subject's brain.

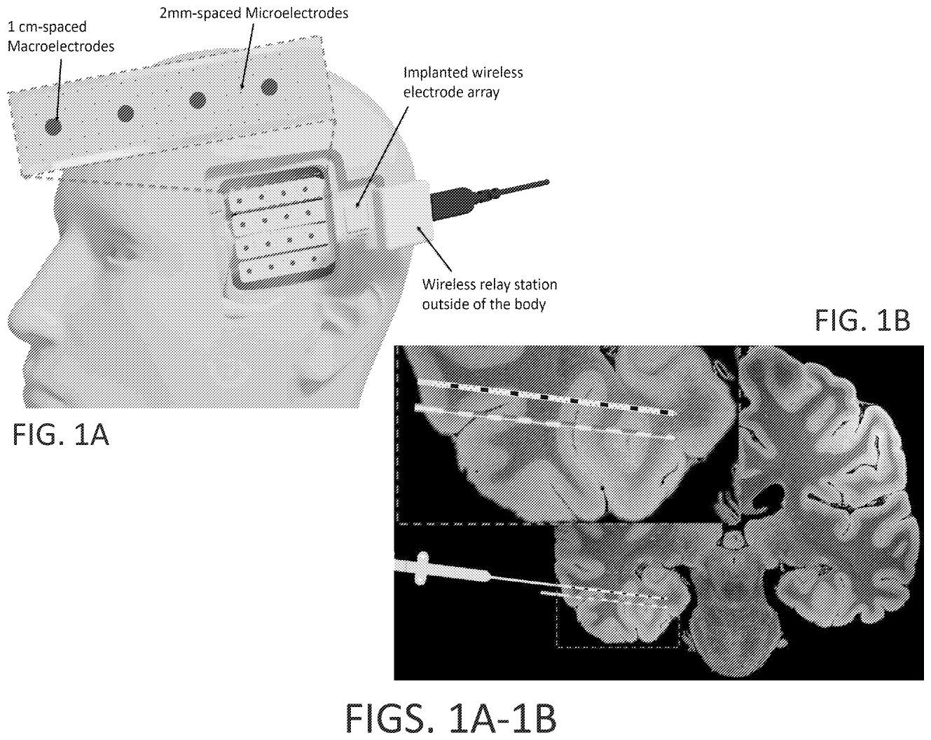

In some embodiments, the electrode array is configured to perform electrocorticography (ECoG) on the subject (FIG. 1A). In some embodiments, the electrode array is configured to perform stereoelectroencephalography (SEEG) on the subject (FIG. 1B). In some embodiments, at least a portion of the electrode array or the signal acquisition component comprise silicone molding. In some embodiments, the majority of the electrode array and the signal acquisition component comprise silicone molding. The silicone coating can have a variety of thicknesses, depending on the clinical application. In some embodiments, the silicone comprises a thickness that is at least about 50 μm, at least about 100 μm, at least about 150 μm, at least about 200 μm, at least about 250 μm, at least about 300 μm, at least about 350 μm, at least about 400 μm, at least about 450 μm, at least about 500 μm, at least about 550 μm, at least about 600 μm, at least about 650 μm, at least about 700 μm, at least about 750 μm, at least about 800 μm, at least about 850 μm, at least about 900 μm, at least about 950 μm, or at least about 1000 μm.

In some embodiments, the silicone comprises a thickness from about 0.1 mm to about 2.0 mm. In some embodiments, the silicone comprises a thickness from about 0.2 mm to about 2.0 mm. In some embodiments, the silicone comprises a thickness from about 0.4 mm to about 1.8 mm. In some embodiments, the silicone comprises a thickness from about 0.6 mm to about 1.6 mm. In some embodiments, the silicone comprises a thickness from about 0.8 mm to about 1.4 mm. In some embodiments, the silicone comprises a thickness from about 1.0 mm to about 1.2 mm. In some embodiments, the silicone comprises a thickness from about 0.5 mm to about 2.0 mm. In some embodiments, the silicone comprises a thickness from about 0.5 mm to about 1.5 mm. In some embodiments, the silicone comprises a thickness from about 0.5 mm to about 1.0 mm.

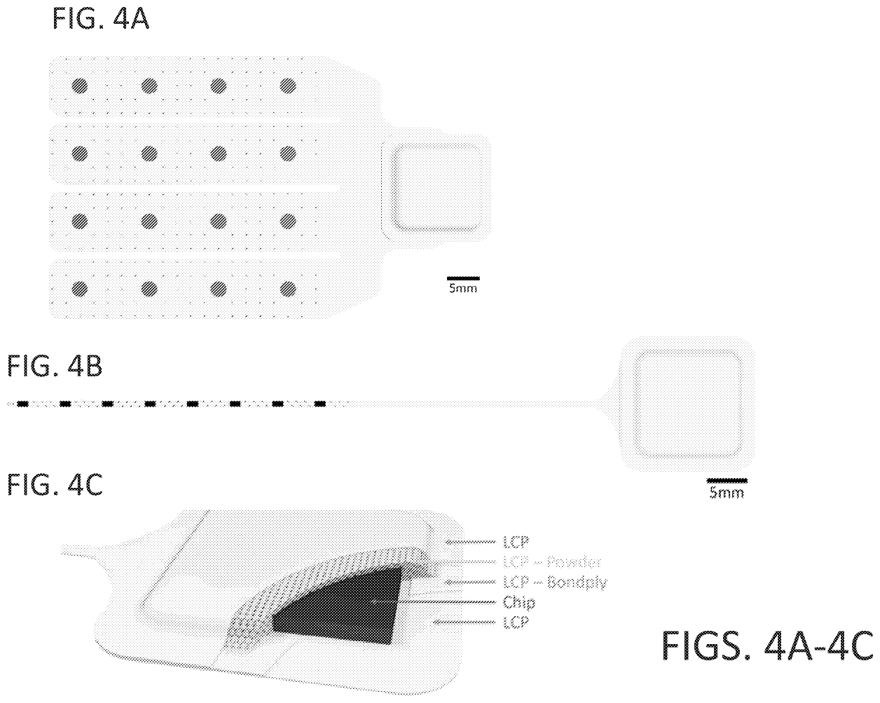

In some embodiments, the polymer-based substrate in which the plurality of microelectrodes and macroelectrodes are uniformly and/or non-uniformly arranged comprises a liquid crystal polymer (LCP). In some embodiments, the polymer-based substrate comprises a liquid crystal polymer (LCP) and any derivatives or variants thereof. As described further herein, the LCP coating can be used to encapsulate the electrode arrays to enhance the overall safety and efficacy of the device. The LCP can be applied to the electrode arrays in singe coatings or multiple coatings, and in any formulation that ultimately provides the desired features (see, e.g., FIG. 4). Generally, LCPs can be divided into two types: liquid-crystalline polymers with mesogen groups in the main-chain and liquid-crystalline polymers with mesogen groups in the side-chain. In both types, gas permeability is dependent on the ordered packing of the polymer chains. Other suitable polymer-based substrates (e.g., polyimides) can also be used with the electrode platforms of the present disclosure, as would be recognized by one of ordinary skill in the art based on the present disclosure. For example, the polymer-bases substrate can include any high-temperature polymers suitable for implantation in a subject.

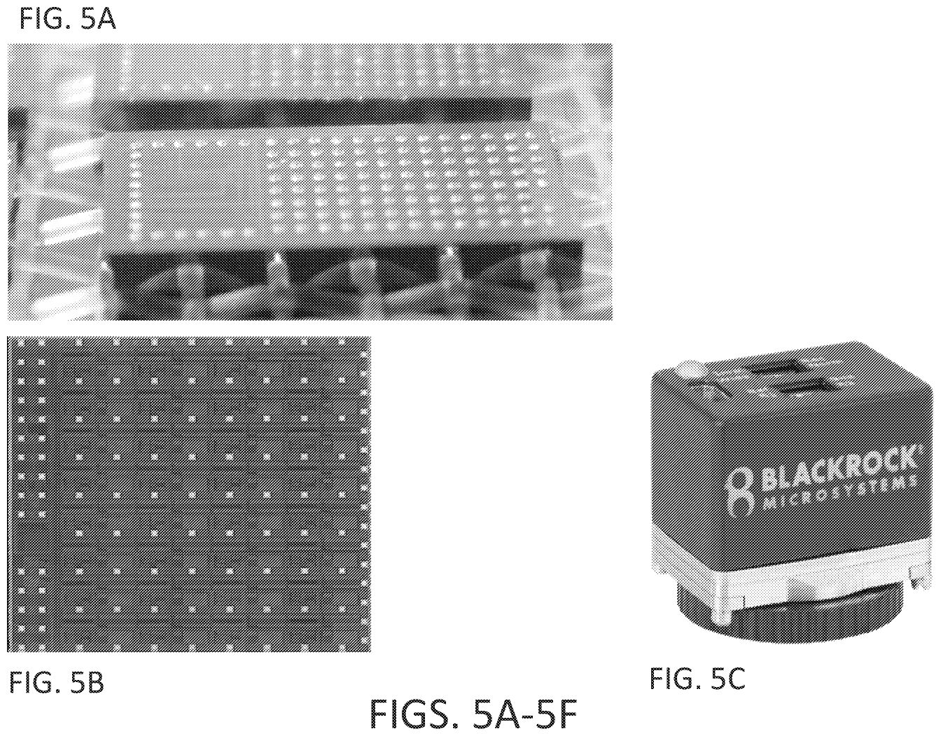

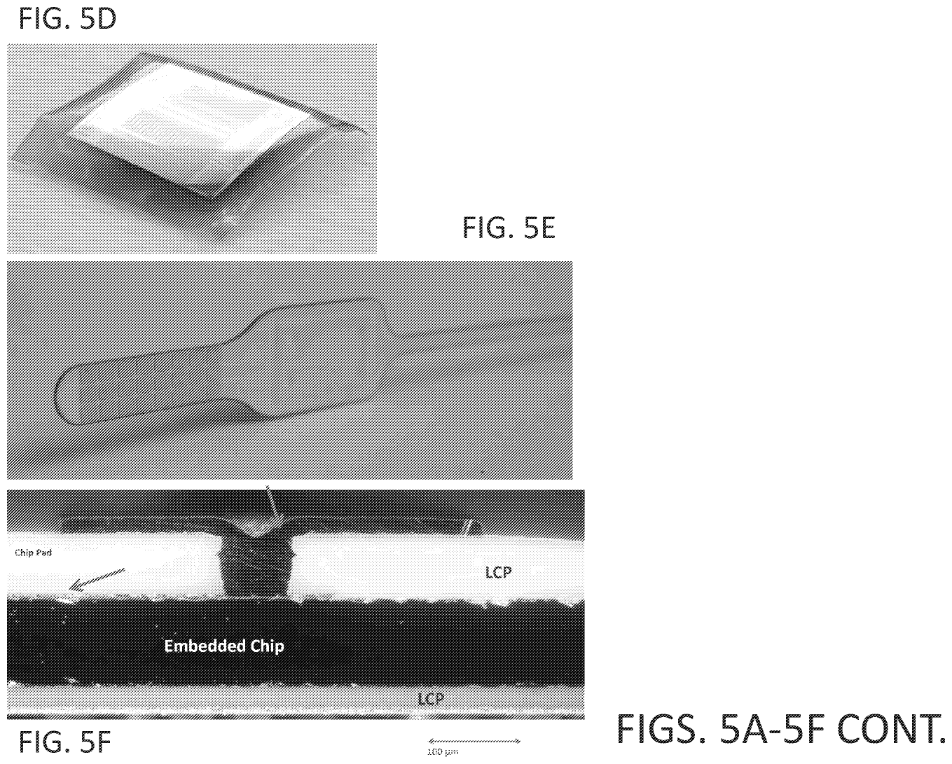

In some embodiments, the ECoG and SEEG devices of the present disclosure further comprise a circuit coupled to the electrode array. In some embodiments, the circuit or circuits are configured to amplify and/or digitize the electrical signals. The circuit can be integrated into the wired or wired versions of the device and have the capability to, for example, amplify, digitize, multiplex and process at least 1024 electrode channels, allow monitoring of electrode health (impedance spectra), identify electrodes damaged/trimmed during surgical implantation and provide automatic mapping with suitable clinical monitoring tools (FIG. 5).

In some embodiments, the signal acquisition component is coupled to a clinical data acquisition system in order to collect data from subjects having the electrode arrays connected to them. In some embodiments, the electrode arrays are implantable and can wirelessly collect and transmit data for long periods of time to neurologists for continuous evaluation. In some embodiments, the signal acquisition component comprises at least one coiled lead comprising a plurality of microwires extending from contacts points on the plurality of microelectrodes and macroelectrodes. In some embodiments, the at least one coiled lead comprises a single lead comprising the plurality of macroelectrodes. In some embodiments, the at least one coiled lead comprises from 2 to 20 leads comprising the plurality of microelectrodes. In some embodiments, the at least one coiled lead comprises from 4 to 18 leads comprising the plurality of microelectrodes. In some embodiments, the at least one coiled lead comprises from 6 to 16 leads comprising the plurality of microelectrodes. In some embodiments, the at least one coiled lead comprises from 8 to 14 leads comprising the plurality of microelectrodes. In some embodiments, the at least one coiled lead comprises from 10 to 12 leads comprising the plurality of microelectrodes. In some embodiments, the at least one coiled lead comprises from 5 to 20 leads comprising the plurality of microelectrodes. In some embodiments, the at least one coiled lead comprises from 5 to 15 leads comprising the plurality of microelectrodes. In some embodiments, the at least one coiled lead comprises from 5 to 10 leads comprising the plurality of microelectrodes.

In some embodiments, the at least one coiled lead comprises from about 10 to about 5000 channels per lead. In some embodiments, the at least one coiled lead comprises at least about 100 channels, at least 200 channels, at least about 300 channels, at least about 400 channels, at least about 500 channels, at least about 600 channels, at least about 700 channels, at least about 800 channels, at least about 900 channels, at least about 1000 channels, at least about 2000 channels, at least about 3000 channels, or at least about 4000 channels. The channels can be active or passive.

In some embodiments, the at least one coiled lead comprises a diameter of at least about 1.0 mm. In some embodiments, the at least one coiled lead comprises a diameter of at least about 2.0 mm. In some embodiments, the at least one coiled lead comprises a diameter of at least about 3.0 mm. In some embodiments, the at least one coiled lead comprises a diameter of about 1.0 mm to about 5.0 mm. In some embodiments, the at least one coiled lead comprises a diameter of about 1.5 mm to about 5.0 mm. In some embodiments, the at least one coiled lead comprises a diameter of about 2.0 mm to about 5.0 mm. In some embodiments, the at least one coiled lead comprises a diameter of about 2.5 mm to about 5.0 mm. In some embodiments, the at least one coiled lead comprises a diameter of about 3.0 mm to about 5.0 mm. In some embodiments, the at least one coiled lead comprises a diameter of about 3.5 mm to about 5.0 mm. In some embodiments, the at least one coiled lead comprises a diameter of about 4.0 mm to about 5.0 mm. In some embodiments, the at least one coiled lead comprises a diameter of about 2.0 mm to about 4.0 mm. In some embodiments, the at least one coiled lead comprises a diameter of about 2.5 mm to about 3.5 mm.

In some cases, the number of leads and array density can increase the degree of adverse events (see, e.g., Arya et al, 2013; Wong et al, 2009). Therefore, the ECoG and SEEG electrode arrays of the present disclosure include embodiments that have reduced numbers of leads. In some embodiments, the present disclosure includes packaging high numbers of leads together in single coils, and/or limiting the size of leads to prevent complications related to leakage of CSF and infection.

In some embodiments, the signal acquisition component comprises a wireless chip (FIG. 4). In some embodiments, the wireless chip is contained within a polymer-based substrate (e.g., LCP-based substrate). In some embodiments, the wireless chip is configured for implantation in the subject, such that it can collect and transmit electrophysiological data from the patient to a neurologist over a given period of time. In some embodiments, the wireless chip is a 65,000 electrode wireless recording chip (FIG. 5).

In some embodiments, the electrode arrays (e.g., ECoG, SEEG, or combinations thereof) of the present disclosure comprise from about 4 to about 500 uniformly or non-uniformly arranged macroelectrodes. Uniform arrangements and non-uniform arrangements generally refer to combinations of both macroelectrodes and microelectrodes configured to enhance the resolution of the electrode arrays to, for example, more accurately identify seizure networks to improve both diagnostic and treatment methods. The electrode arrays can comprise combinations of macroelectrodes and microelectrodes embedded in an LCP-based substrate that are arranged in either uniform patterns, non-uniform patters, or combinations of uniform and non-uniform patterns.

In some embodiments, the macroelectrodes comprise diameters from about 1 mm to about 10 mm. In some embodiments, the macroelectrodes are spaced from about 0.1 cm to about 5 cm apart from each other. In some embodiments, the macroelectrodes are spaced from about 0.2 cm to about 5 cm apart from each other. In some embodiments, the macroelectrodes are spaced from about 0.3 cm to about 5 cm apart from each other. In some embodiments, the macroelectrodes are spaced from about 0.4 cm to about 5 cm apart from each other. In some embodiments, the macroelectrodes are spaced from about 0.5 cm to about 5 cm apart from each other.

In some embodiments, the electrode array comprises from about 100 to about 10,000 uniformly or non-uniformly arranged microelectrodes. In some embodiments, the microelectrodes comprise diameters from about 1 μm to about 1 mm. In some embodiments, the microelectrodes are spaced from about 100 μm to about 5 mm apart from each other. In some embodiments, the microelectrodes are spaced from 100 μm to 4 mm apart from each other. In some embodiments, the microelectrodes are spaced from about 100 μm to about 3 mm apart from each other. In some embodiments, the microelectrodes are spaced from about 100 μm to about 2 mm apart from each other. In some embodiments, the microelectrodes are spaced from about 100 μm to about 1 mm apart from each other. In some embodiments, the microelectrodes are spaced from about 200 μm to about 5 mm apart from each other. In some embodiments, the microelectrodes are spaced from about 300 μm to about 5 mm apart from each other. In some embodiments, the microelectrodes are spaced from about 400 μm to about 5 mm apart from each other. In some embodiments, the microelectrodes are spaced from about 500 μm to about 5 mm apart from each other. In some embodiments, the microelectrodes are spaced from about 600 μm to about 5 mm apart from each other. In some embodiments, the microelectrodes are spaced from about 700 μm to about 5 mm apart from each other. In some embodiments, the microelectrodes are spaced from about 800 μm to about 5 mm apart from each other. In some embodiments, the microelectrodes are spaced from about 900 μm to about 5 mm apart from each other. In some embodiments, the microelectrodes are spaced from about 1 mm to about 5 mm apart from each other.

In some embodiments, the electrode array provides at least about a 2-fold increase in spatial sampling resolution compared to an array comprising only macroelectrodes. In some embodiments, the electrode array provides at least about a 4-fold increase in spatial sampling resolution compared to an array comprising only macroelectrodes. In some embodiments, the electrode array provides at least about a 6-fold increase in spatial sampling resolution compared to an array comprising only macroelectrodes. In some embodiments, the electrode array provides at least about a 8-fold increase in spatial sampling resolution compared to an array comprising only macroelectrodes. In some embodiments, the electrode array provides at least about a 10-fold increase in spatial sampling resolution compared to an array comprising only macroelectrodes. In some embodiments, the electrode array provides at least about a 15-fold increase in spatial sampling resolution compared to an array comprising only macroelectrodes. In some embodiments, the electrode array provides at least about a 20-fold increase in spatial sampling resolution compared to an array comprising only macroelectrodes. In some embodiments, the electrode array provides at least about a 25-fold increase in spatial sampling resolution compared to an array comprising only macroelectrodes.

### 3. WIRELESS IMPLANTABLE ELECTRODE ARRAYS

As is known in the art, the best treatment option for pharmacologically resistant epilepsy is resective surgery, yet only 50% of surgical patients are seizure free. This lack of success may be due to poor monitoring of patient-specific seizure networks as current electrographic devices are limited in temporal and spatial sampling. Furthermore, these invasive studies are performed in an atypical state (with the patient hospitalized, off medication, etc.) and only over brief periods of time. This may lead to incomplete localization and consequently poorer surgical outcomes. To better identify patient-specific seizure networks, a novel implantable wireless electrode array was developed that allows high-resolution measurement from thousands of sensors over large areas of the brain. Wireless data and power transmission in a small implanted package eliminates the need for passing connection wires through the skin, as is the current standard for invasive studies. By making the array fully implanted and wireless, the monitoring period can be extended from a number of days to several months. This enables patients to return home for remote seizure monitoring, rather than being tethered to an inpatient monitoring system. This paradigm shift in epilepsy pre-surgical evaluation will dramatically improve surgical outcomes.

In accordance with these embodiments, the present disclosure provides a functional wireless electrode array that enables long-duration seizure monitoring, thereby improving the ability to detect patient-specific seizure networks, and providing for more effective surgical resections. The wireless electrode arrays of the present disclosure include combinations of macroelectrodes and microelectrodes with 25× higher spatial resolution than current clinical standard arrays. This increase in electrode density enables detection of high frequency oscillations, microseizures, and other micro-scale electrographic phenomena. These biomarkers are poorly sampled or missed entirely with existing technology and could enable dramatic improvements in epilepsy treatment. As described further herein, the wireless implantable electrode arrays of the present disclosure permit high-density, long-term electrographic studies (6-12 months) of patient seizure networks. These features will improve the characterization of patient seizure networks, thereby enabling more effective applications of current and future surgical treatments.

Embodiments of the present disclosure include three core technology advantages: low cost manufacturing, near-hermetic circuit packaging, and a fully-integrated, wireless system on chip (SOC). These features allow for the integration of wireless recording systems with thin-film (TF) electrode arrays, and facilitate the implantation of wireless arrays to evaluate long-term recording capabilities. The recording and stimulating technology described herein, which includes up to ˜65,000 electrodes, is integrated with wireless technology to enable longer term and more accurate monitoring of patient-specific seizure networks. These embodiments can be used with concurrent optogenetic stimulation and behavioral recording to assess signal specificity, and to monitor seizure related activity in humans (e.g., interictal activity, microseizures, and high-frequency oscillations). These wireless electrode array platforms will enable more accurate neurosurgical planning of patient-specific seizure networks, leading to dramatically improved outcomes.

In accordance with these embodiments, the fully wireless implantable electrode arrays of the present disclosure provide high-density, long-term recordings of seizure networks outside the epilepsy monitoring unit in order to guide more effective medical management and surgical treatment through a naturalistic sampling of each patient's individual seizure network. Fully implantable wireless electrode arrays of the present disclosure will replace the current standard of care of wired ECoG and SEEG electrodes. Patients undergoing invasive intracranial electrophysiological studies could be first evaluated with this technology. These implantable arrays will wirelessly collect data for up to a year and stream this data to neurologists for continuous evaluation. These devices include neurostimulation to enable clinicians to test excitability in individual patient seizure networks using cortico-cortical evoked potentials (CCEPs) or to test the effectiveness of stimulating specific areas to prevent seizures. The combination of stimulation and recording capability will help inform future neuromodulation-based therapies. After 6-12 months, the devices can be removed and surgical treatment delivered.

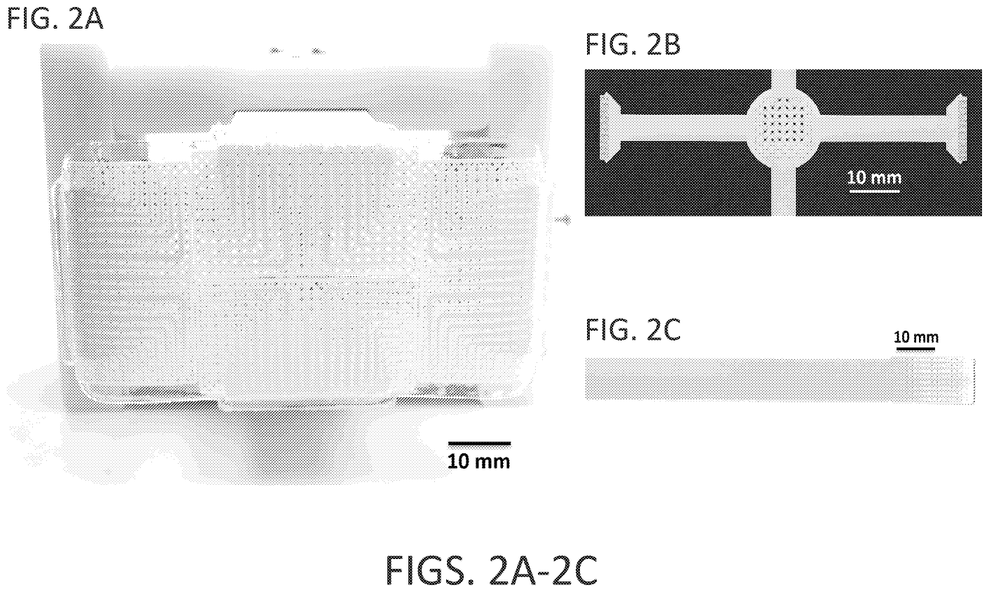

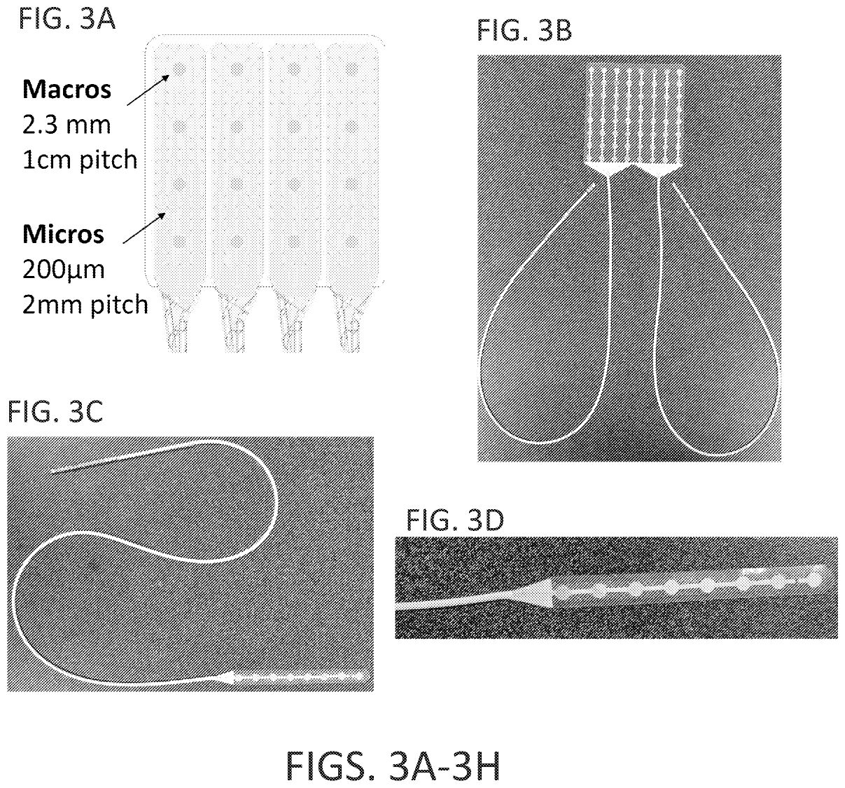

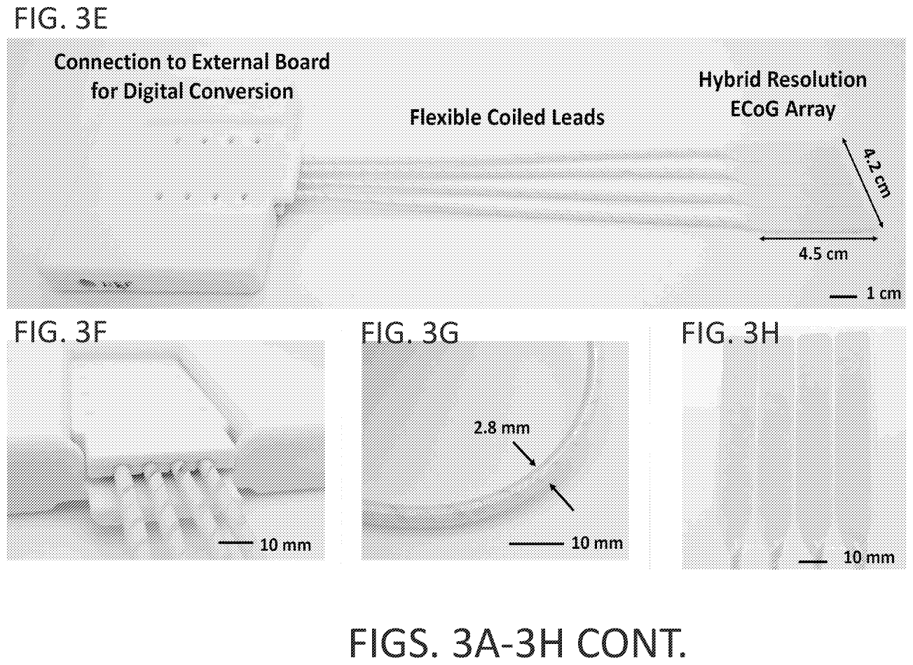

As described further herein, six generations of wired surface TF electrode arrays have been fabricated using LCP (see, e.g., FIGS. 2 and 3). These devices enabled 25× to 177× higher resolution sampling from the brain with an electrode spacing (center-to-center distance) ranging from 750 μm to 2 mm. Using LCP instead of other materials, such as polyimide, offers several advantages. LCP is long-lasting, with water permeability about 25× lower than polyimide substrates, dramatically extending the reliability and lifetime of implanted arrays. In one embodiment, the electrode arrays are formed from two 25 μm-thick LCP sheets fused into a single LCP layer using extreme heat and pressure, yielding a total TF device thickness of just over 50 μm. The LCP devices are molded in silicone to provide soft, rounded edges. In one embodiment, the molded device is about 200 μm thick, less than 25% the thickness of standard subdural surface clinical grid (CG) electrode arrays manufactured by AdTech (850 μm). Since there is a single LCP layer, the LCP-TF electrode cannot delaminate and will last over 5 years when implanted. By contrast, polyimide electrode arrays rely on adhesion between layers and delaminate, sometimes lasting <1 year. Fiducial markers are also integrated to allow electrode placement verification by using a CT scan after implantation.

The electrode arrays of the present disclosure include a unique design featuring round leads, which neurosurgeons consider acceptable for chronic implantation. The planar TF LCP leads are molded into helically wound cables (<3 mm diameter, without sharp edges) that allow longitudinal elongation up to 15% strain (FIG. 3). Individual Au wiring layers and interlayer connections in the LCP are lithographically patterned. The TF electrodes are produced by additive manufacturing rather than conventional semiconductor processing, making them less expensive and the project economically feasible and eventually commercially competitive.

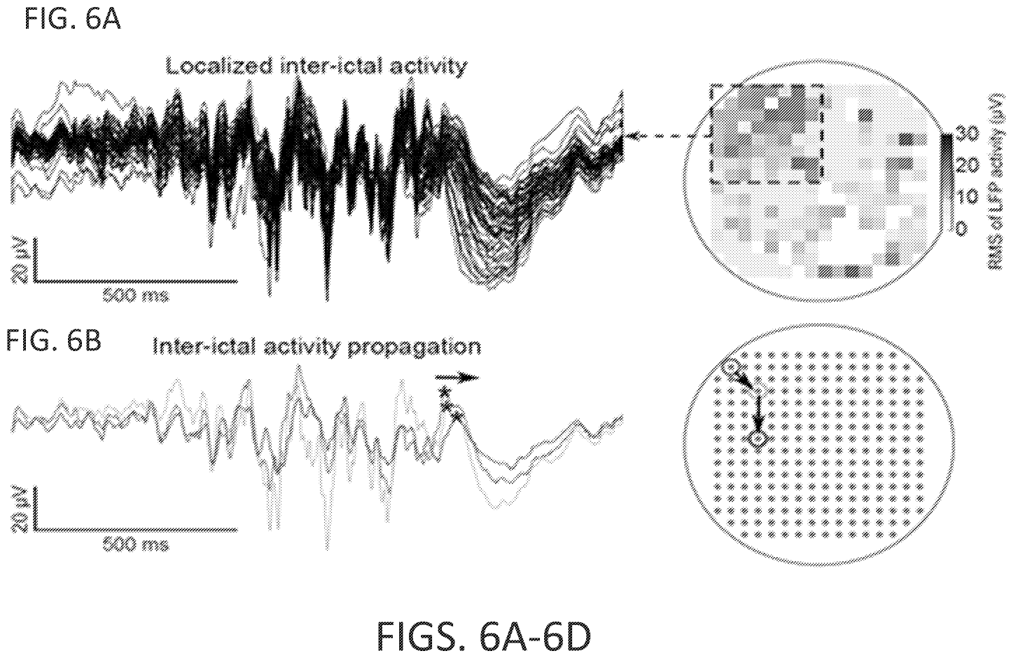

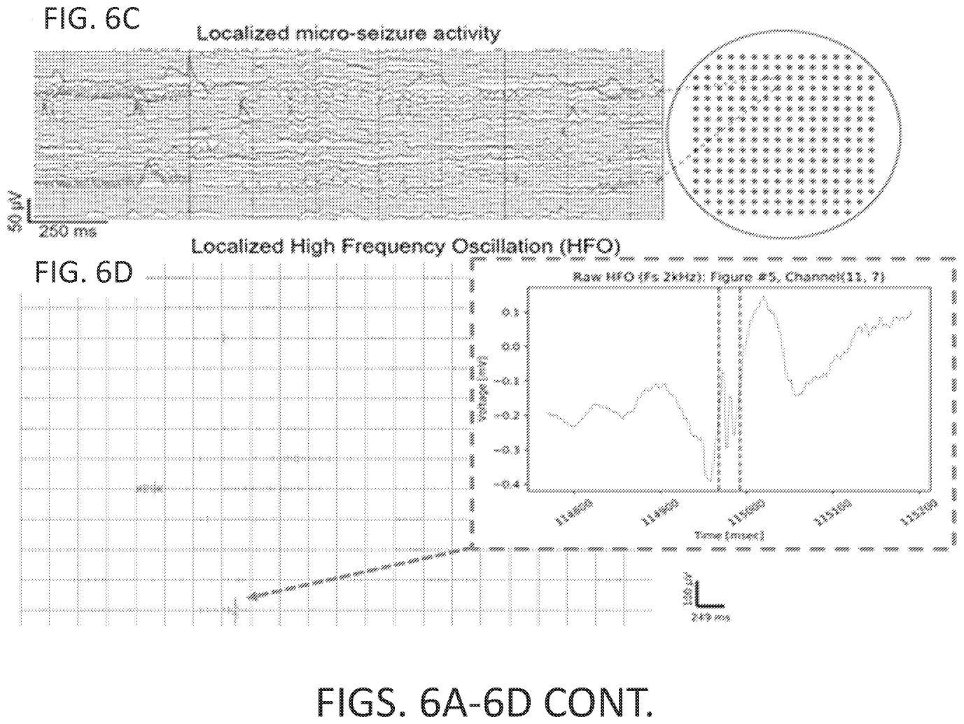

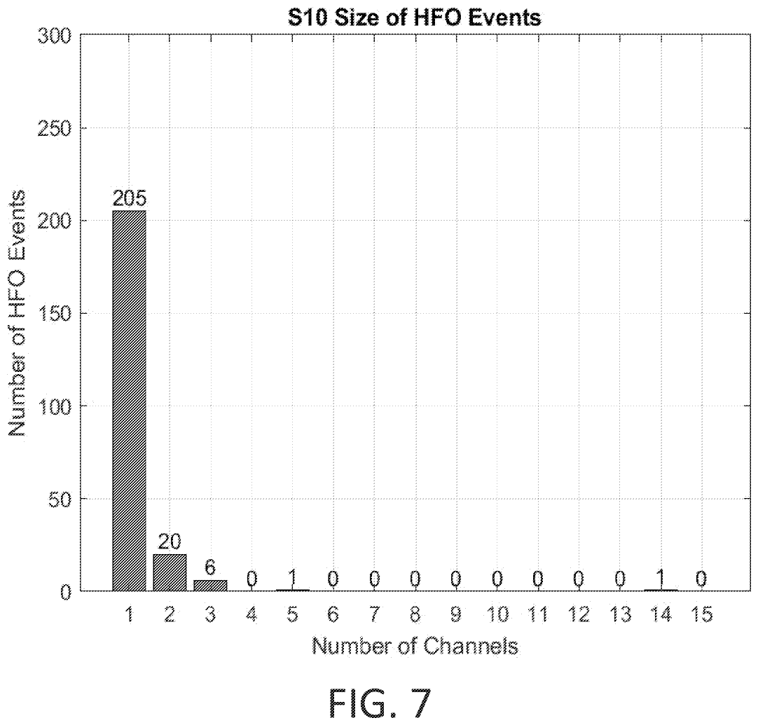

The electrode arrays of the present disclosure have been thoroughly tested in chronic implants in rats for over a year and in nonhuman primates for >6 months. Several different generations of the arrays have been tested intraoperatively in 10 patients. High quality neural signals have been collected in these experiments, including propagating interictal discharges (IIDs), high frequency oscillations (HFOs), and microseizures (FIG. 6). Importantly, microseizures were observed occurring on just two electrodes spaced 750 μm apart, and were not visible on neighboring electrodes. Similarly, 88% of all detected HFOs occurred on a single channel, and were not detected on neighboring electrodes 1.7 mm away (FIG. 7). These observations motivate the use of high-density electrode arrays to sample microscale phenomena that are undetectable by conventional arrays. As described further herein, high-density μSEEG electrodes have also been fabricated (FIG. 4). A wireless recording integrated circuit (IC) (see, e.g., FIG. 5) and an external wireless relay station have also been fabricated and tested.

In some embodiments, the electrode arrays of the present disclosure include wiring that is composed of pure gold traces that are about 5 μm thick, thicker than traditional TF arrays which are often <500 nm thick. The thick Au layer reduces the resistance of the wire traces and improves their mechanical reliability and durability to surgical handling. Preliminary mechanical bend testing of the electrode leads was performed. The leads were successfully bent by 90° for over 47,000 cycles without breaking any wire traces (FIG. 8). Other experiments included performing bend testing on devices with thinner gold (e.g., about 500 nm) to a 1-mm bend radius (nearly folding) and achieved 6,000-12,000 bend cycles before failure. The mechanical properties of the lead are flexible despite having more channels. The typical microelectrode trace resistance was measured as 2.7 Ω/cm, with a 40-cm cable having a trace resistance ˜110Ω, which is an order of magnitude below the average microelectrode impedance of 2.8±0.8 kΩ at 1 kHz. The electrode contact yield (number of functional microelectrode contacts per device) is about 99.5%. Wider traces were used for the macroelectrodes to achieve 100% yield and decreased trace resistance, which is important to reduce the required stimulator compliance voltage. All electrode contacts were plated with platinum iridium to reduce impedance.

The TF electrode arrays of the present disclosure were immersed in saline at 60° C., resulting in a 5× accelerated aging (AA) rate versus body temperature (37° C.). These AA studies demonstrate that the electrode arrays will have a lifetime of at least 5 years at 37° C., and up to 10 years.

The difference in Young's modulus between neural tissue and electrode materials is typically large (>10⁵× for polymer electrodes, >10⁸× for silicon probes). Ideal materials would have a Young's modulus and Poisson ratio similar to those of neural tissue. Microglia move from softer to stiffer surfaces and exert larger forces on stiffer surfaces. From a clinical perspective, the bending stiffness of the electrode and the resulting forces acting on the tissue are important for determining the tissue response. The force exerted by the device on the brain must be minimized. Compliance can be improved in several ways: by using materials with lower elastic modulus, reducing the cross-sectional area, and increasing the length of the device. Thinner, more flexible electrode arrays reduce the force exerted on the brain and potentially improve surgical safety, patient tolerance, and data quality. More flexible devices also reduce the potential for venous infarction, inflammatory changes, and injury to the cortex.

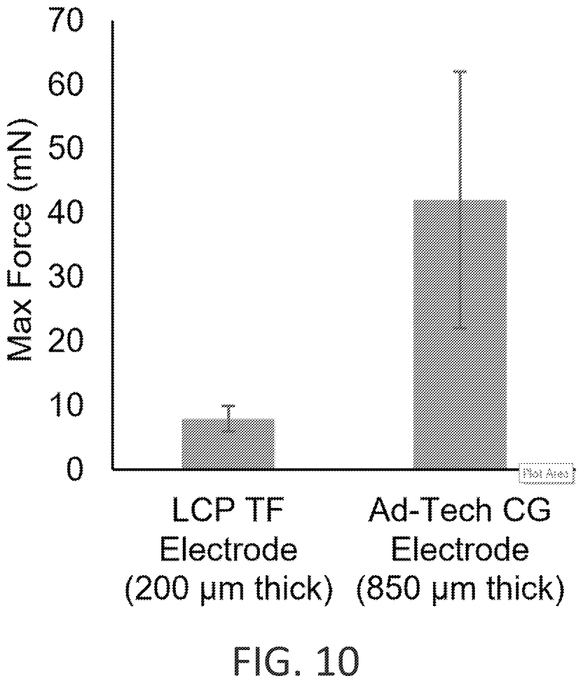

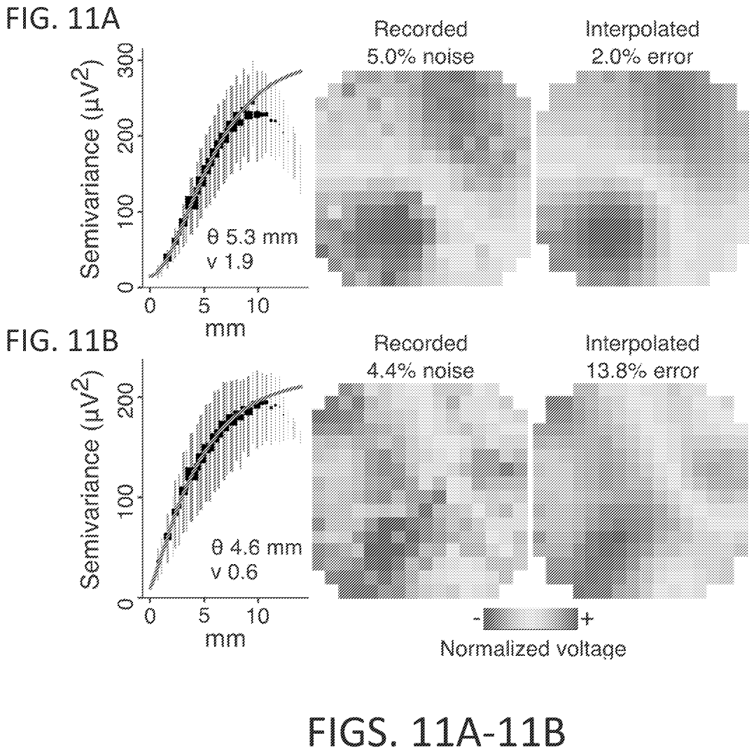

The maximum possible force exerted on brain tissue from the TF μECoG electrode arrays under flexion is more than 5× lower than that of the commercial CG from AdTech (7.9±2 mN vs. 42±20 mN). The LCP TF μECoG electrode is much more flexible than the thicker AdTech CG electrode and exerts less force under flexion (FIG. 10). This flexibility and reduced bending force will translate to less force exerted on the brain.

As described further herein, the wireless IC can be combined with the LCP-based ECoG and SEEG electrode arrays to create active μECoG and μSEEG electrode arrays. Importantly, this packaging technique enables high-density feedthroughs supporting>1000 electrodes, is extremely low-cost, and durable enough to remain intact for the proposed 6-12 month implantation period (see, e.g., FIG. 5). The TF electrode technology and wireless circuits of the present disclosure enable high-resolution sampling of microscale neural activity. Current generation implantable wireless systems such as NeuroPace and Medtronic RC+S include only four channels of wireless recording. In contrast, TF LCP-based electrode arrays of the present disclosure include a 65,536-channel wireless recording and stimulating IC (FIG. 5), and an external wireless relay station. For example, this IC can be combined with LCP-based electrodes to create a wireless μECoG electrode array with 320 electrodes that can sample from a larger area of the brain than the IC itself could (40×40 mm vs. 8×8 mm). The electrode arrays of the present disclosure include 320 channels of wireless recording (FIG. 1), and multiple arrays can be implanted simultaneously. The IC consumes<32 mW of power and will not get warmer than 1° C. above body temperature when used on the surface of the brain. The IC will be contained in a package that will spread this heat over a larger area, and the IC will be located on top of the skull, which is less sensitive to heat than the brain. This IC can support future increases in the number of electrodes without significant modification.

### 4. SYSTEMS

Embodiments of the present disclosure also include an electroencephalography (EEG) system comprising an electrode array having a plurality of microelectrodes and macroelectrodes uniformly or non-uniformly arranged in a polymer-based substrate, a signal acquisition component coupled to the electrode array configured to collect and transmit electrical signals obtained from a subject's brain, and a clinical data acquisition system.

In some embodiments, the electrode array is configured to perform electrocorticography (ECoG) on the subject. In some embodiments, the electrode array is configured to perform stereoelectroencephalography (SEEG) on the subject. In some embodiments, the system comprises an electrode array configured to perform electrocorticography (ECoG) on the subject, and an electrode array configured to perform stereoelectroencephalography (SEEG) on the subject (combination ECoG/SEEG electrode array). In some embodiments, the device further comprises a circuit coupled to the electrode array and configured to amplify and/or digitize the electrical signals. In some embodiments, the signal acquisition component comprises at least one coiled lead comprising a plurality of microwires extending from contacts points on the plurality of microelectrodes and macroelectrodes.

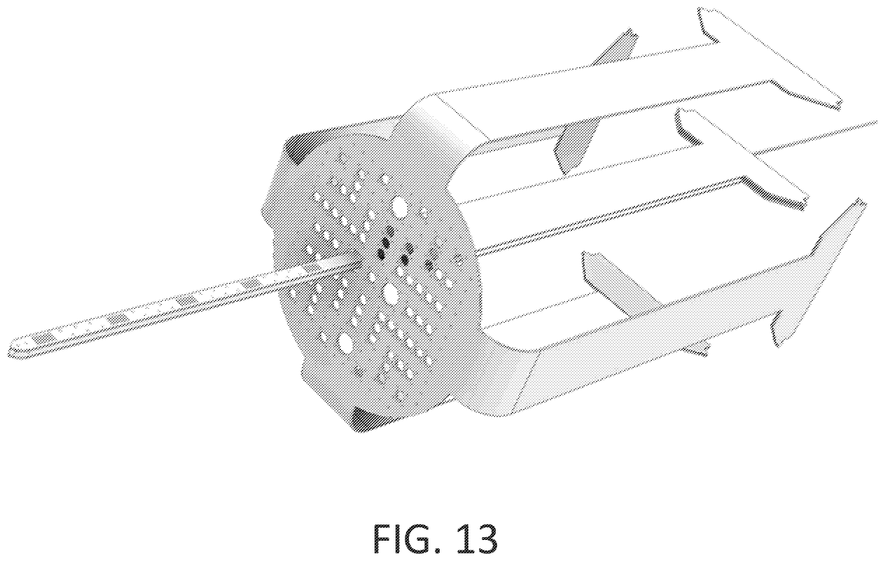

Embodiments of the present disclosure also include an electrocorticography (ECoG)-stereoelectroencephalography (SEEG) combination device comprising an ECoG component and an SEEG component (FIG. 13). In accordance with these embodiments, the combination ECoG/SEEG device includes an electrode array comprising a plurality of microelectrodes and macroelectrodes uniformly or non-uniformly arranged in a polymer-based substrate, and a signal acquisition component coupled to the electrode array configured to collect and transmit electrical signals obtained from a subject's brain; and an SEEG component having an electrode array comprising a plurality of microelectrodes and macroelectrodes uniformly or non-uniformly arranged in a polymer-based substrate, and a signal acquisition component coupled to the electrode array configured to collect and transmit electrical signals obtained from a subject's brain.

In accordance with these embodiments, the ECoG/SEEG combination device of the present disclosure enable neural recordings from both cortical grid (ECoG) and stereotactically inserted depth electrodes (SEEG), which can provide enhanced neural recordings and diagnostic capability than either device alone. Depending on the characteristics of the subject being diagnosed and/or treated, as well as the characteristics of the particular disease/disorder, the ECoG/SEEG combination device of the present disclosure can minimize diagnostic errors due to, for example, insufficient sampling of the neutral activity. Additionally, the ECoG/SEEG combination device of the present disclosure provides the advantages of being able to measure neural activity in deeper brain structures using a lower risk profile of SEEG, with the more widespread cortical sampling of ECoG in the same patient.

### 5. EVALUATION AND TREATMENT

Drug-resistant epilepsies affect nearly 1 million Americans and are associated with high morbidity and mortality. Invasive surgery remains the primary treatment for many of these patients but success in long-term seizure freedom appears highly variable. Patient comfort and perceived risks compared to Anti-Epileptic Drugs (AEDs) lead to only 1-2% of the >300,000 patients with drug resistant epilepsies in the US being treated with surgical intervention as last resort treatment at one of ca. 180 centers in the US. Average time for referral is 15-16 years. About 39% of patients are self-referred, 36% of patients had been actively advised against surgery in favor of AEDs. This is fundamentally different from countries where surgery is considered an effective and preferred treatment. Diagnostic electrode studies that strongly guide decisions on resection boundaries expose patients to high surgical risk including inflammation, pain, and extended hospital recovery, which can adversely affect long-term outcomes. Existing diagnostic solutions (ECoG, SEEG) have limitations: the gold standard diagnostic technology for surgical intervention has been intracranial EEG for decades. Early SEEG electrodes were largely replaced with ECoG electrodes starting in the 1950s which allowed covering larger areas of cortex with more contacts. More recently, SEEG use has resurged again due to the advent of robotic surgical methods, reduced inflammation and better patient comfort compared to ECoG. Neither technology has changed in the past 30 years and both lack the ability to provide higher precision diagnostics and localization of seizure foci.

Embodiments of the present disclosure include high-density, high-channel-count wireless electrode arrays, which facilitate investigation of the microelectrophysiology of epilepsy and large scale seizure networks, over long periods of time, outside of the epilepsy monitoring unit. Additionally, the ECoG and SEEG electrode arrays of the present disclosure provide at least a 25× increase in sampling resolution will also allow for better seizure network localization and provide higher quality data for surgical resection. Additionally, the wireless electrode arrays of the present disclosure can be implanted into subject and allowed to remain there for up to a year; this allows for the incorporation of neurostimulation protocols into treatment plans, which is not possible in current electrode platforms. The electrode arrays of the present disclosure can be used to investigate different responsive stimulation treatment paradigms, thereby allowing clinicians to better target areas of a subject's brain and determine if the treatment is working. The wireless arrays of the present disclosure also allow patients to continue their lives at home, relatively unhindered, which greatly improves quality of live.

Embodiments of the ECoG and SEEG electrode arrays and systems of the present disclosure are useful for various therapeutic, prophylactic, and diagnostic purposes. For example, the ECoG and SEEG electrode arrays and systems described herein can be used to monitor neural activity in a subject, which can include recording or mapping neural activity to diagnose a disease or disorder, as well as modulating neural activity in a subject, which can include stimulating neural activity to treat a disease or disorder. In some embodiments, monitoring neural activity includes recording and processing ECoG and/or SEEG data for functional mapping in order to identify neurological dysfunction, which can be indicative of a disease or condition. In some embodiments, electrical stimulation via the macroelectrodes and/or microelectrodes allows for the mapping of various parts of the brain to aid in the identification or diagnosis of neurological dysfunction.

In some embodiments, electrical stimulation via the macroelectrodes and/or microelectrodes allows for the modulation of neural activity in the subject to treat a disease or disorder. Stimulation through ECoG and/or SEEG electrode arrays, such as those described herein, can be used to target an underlying region of interest to treat a disease or disorder. In some embodiments, ECoG and/or SEEG stimulation can be used clinically as a treatment to reduce seizures or neuropathic pain, to support recovery and plasticity in stroke, and to provide sensory feedback for bidirectional brain-computer interfaces, among other treatments.

In accordance with the above, embodiments of the present disclosure include a method of evaluating a subject for a neurological impairment. The method includes recording electrical signals in a portion of a subject's brain using the ECoG/SEEG electrode array devices described herein, and evaluating the subject based on the recorded electrical signals. In some embodiments, the neurological impairment comprises a seizure. In some embodiments, the neurological impairment comprises a epilepsy.

In some embodiments, evaluating the subject comprises determining the presence or absence of the neurological impairment, and/or determining the severity of the neurological impairment. In some embodiments, evaluating the subject comprises identifying means for treating the neurological impairment. In some embodiments, the method further comprises treating the neurological impairment in the subject. As would be recognized by one of ordinary skill in the art based on the present disclosure, treatment can include, but is not limited to, pharmacological intervention, surgical intervention, and/or electrophysiological intervention.

Embodiments of the present disclosure also include a method for treating a subject having or suspected of having a neurological impairment. In accordance with these embodiments, the method includes recording electrical signals in a portion of the subject's brain using an electrode array comprising a plurality of microelectrodes and macroelectrodes uniformly or non-uniformly arranged in a polymer-based substrate, and a signal acquisition component comprising a wireless chip coupled to the electrode array configured to collect and transmit electrical signals obtained from a subject's brain. Additionally, the method includes administering an electrical pulse to at least a portion of the subject's brain to treat the neurological impairment.

For example, in some embodiments, the wireless implantable ECoG and SEEG electrode arrays of the present disclosure include recording and stimulating capability, which enables the development of novel therapeutic approaches. For example, the wireless implantable ECoG and SEEG electrode arrays of the present disclosure include can be used for the treatment of seizures and epilepsy, for controlling remote prostheses, for evoking somatosensory sensation, for deep brain stimulation for Parkinson's Disease, and to administer responsive neuro-stimulation (e.g., for treating epilepsy).

In some embodiments, such as when an array is implanted for at least 28 days, the devices of the present disclosure can be used for responsive neurostimulation for epilepsy, deep brain stimulation for movement disorders (e.g., Parkinson's, tremor, etc.), epilepsy, pain control/relief, satiety/obesity and psychiatric/mood disorders (e.g., OCD/tics; see, e.g., Graat et al., 2017). In other embodiments, the wireless implantable ECoG and SEEG electrode arrays of the present disclosure can be used for reconstructing deficits like vision, hearing, smell, touch, and potentially cognition (see, e.g., Kundu et al., 2018). The augmentation of normal function is also possible, such as seeing infrared, echo location, synesthetic increases, restoration, and the like. Additionally, the wireless implantable ECoG and SEEG electrode arrays of the present disclosure can include non-therapeutic applications, including for example, to connect the brain to the internet of things, the brain to other machine such as cars, smart home, robots, fighter jet/airplane, heavy machinery, and the like. The device could receive as well as transmit or receive information from or to the brain.

### 6. MANUFACTURING

Embodiments of the present disclosure include integrated wireless recording systems with TF electrode arrays. In accordance with these embodiments, the electrode arrays include wireless recording chips that supports recording and stimulating up to 65,536 electrodes (FIG. 5) with high-density liquid crystal polymer (LCP) μECoG and μSEEG electrode arrays (FIGS. 1-4). The arrays can be wired (see, e.g., FIG. 3) or wireless (see, e.g., FIG. 4), in which a short cable from the array extends to a bond area for the wireless chip. An LCP cover is thermally-bonded on top of the chip to protect it while implanted.

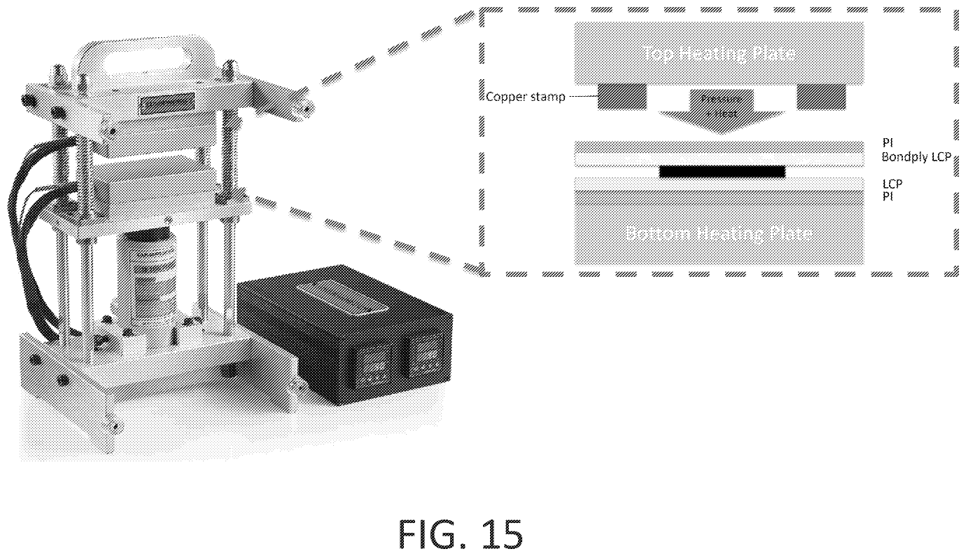

To test the efficacy of the LCP packaging, a heat-press setup was built to thermally fuse layers of LCP film to form the wireless IC package (FIG. 15). Exemplary packages included magnesium (Mg) metal strips for testing for water intrusion and copper (Cu) conductors for measuring leakage current. Packages were tested using accelerated aging by soaking them in phosphate buffered saline (PBS) heated to 60° C., an approximately 5× acceleration over body temperature (37° C.). Mg was used as a highly sensitive optical indicator of encapsulation failure. The thin Mg metal dissolves in the presence of water. Light was shined through the LCP package to inspect for Mg dissolution. The Cu metal was also connected to a 3V DC bias and measure leakage current to a Pt reference wire outside the package. A picoammeter was used to test for leakage currents. A leakage current >1 μA indicated sealing failure. Additional sealing mechanisms can include filling the LCP package with epoxy or applying an additional layer of conformal coatings to protect the electronics prior to sealing them inside the LCP package. Conformal coatings of alumina and parylene, silicon dioxide, or silicon carbide can also be used.

Electrically functional LCP packages were also fabricated with integrated test circuits. Gold (Au) circuit traces were deposited onto LCP substrates. A bare-die, commercial IC, such as the CC430 (Texas Instruments) was then flip-chip bonded onto the LCP substrate. The ICs were successfully bonded to the LCP substrate and electrical functionality was verified. The LCP package cover was then bonded to the LCP substrate, as described further herein. The packaged IC was then soak tested and it was verified that the IC remained fully functional and that the package did not develop significant leaks over the soak testing period in 60° C. saline.

Additionally, mechanical bend testing of the LCP electrode leads was performed. The leads were successfully bent by 90° over 47,000 cycles without breaking any wire traces (FIG. 8). Bend testing was also performed on a device with thinner gold (500 nm) to a 1 mm bend radius (nearly folding) and achieved 6,000-12,000 bend cycles before failure.

In some cases, wire-bonding can be used to connect the ICs to the LCP substrate or bonding using anisotropic conducting film (ACF). Another option involves having the ICs packaged into a standard ball-grid array (BGA) package and then solder the BGA package to the LCP substrate. This process is similar to the standard used in the industry.

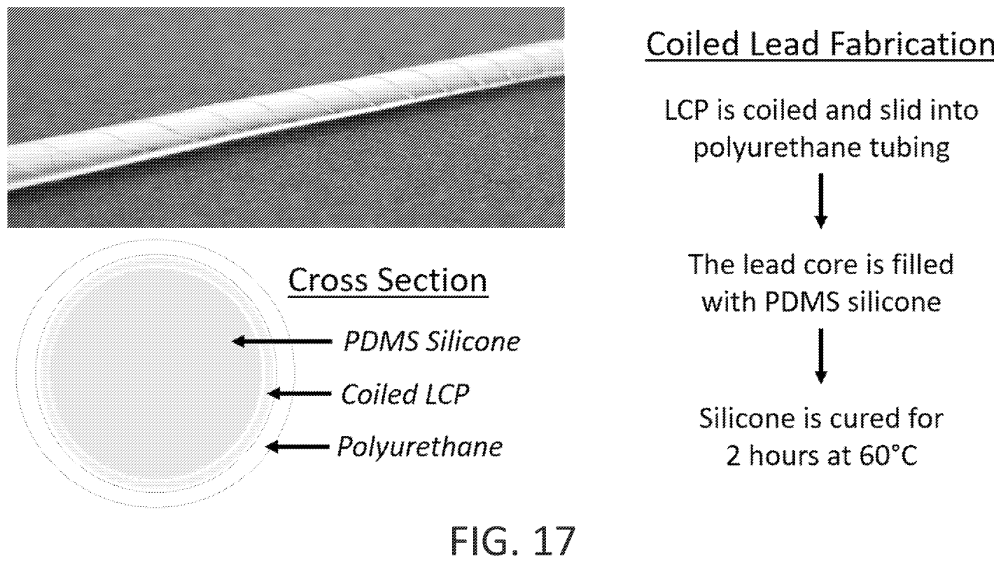

In some embodiments, a coiled lead(s) used as part of the ECoG and SEEG electrode arrays of the present disclosure (see, e.g., FIG. 3) comprise silicone molding, which provides additional mechanical strength and protection. In some embodiments, thin-film manufacturing can lead to sharp edges, which can injure the cortical tissue during implantation. To prevent damage to the brain, the edges of the arrays can be molded in soft, flexible silicone. Recording performance remains unaffected following this molding method. FIG. 17 includes representative diagrams of a coiled lead design for an electrode array, according to one embodiment of the present disclosure. Micro wires from the array contacts are fully encased in the liquid crystal polymer (LCP) substrate and extend from the array in the form of flat, flexible strips. These flat strips are coiled and adhered within a silicone/polyurethane structure to form the leads. This results in round, mechanically sturdy leads which can be threaded out of the skull and skin to connect the implanted array with an external acquisition system. The diameter of the coiled lead is chosen to ensure that the implantation site remains sufficiently sealed to minimize communication between the intracranial and extracranial sites in order to reduce risk of post-operative morbidity and infection as well as to reduce postoperative scarring.

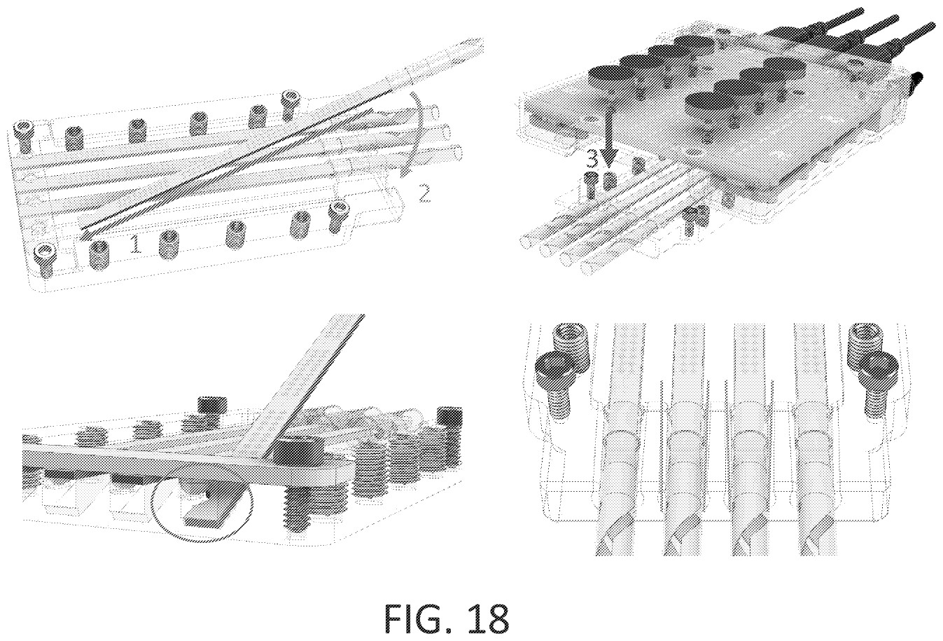

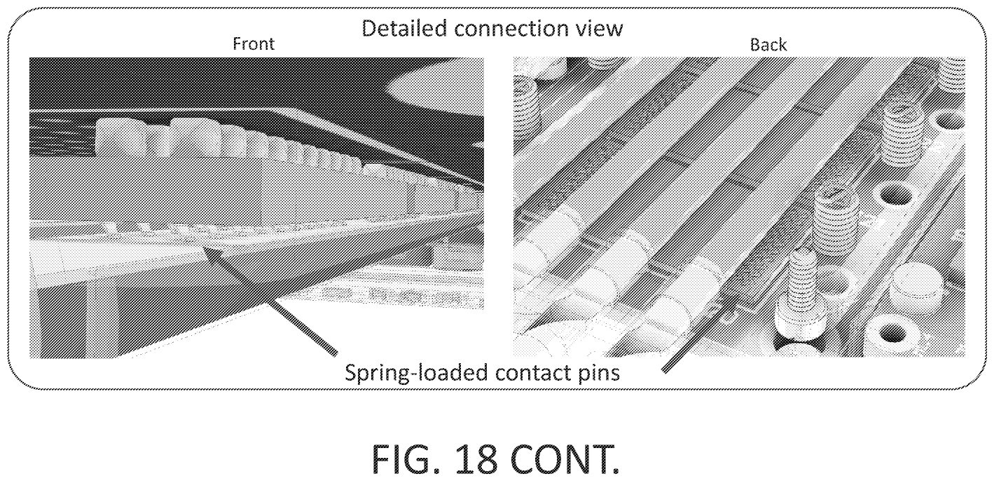

Additionally, FIG. 18 includes representative diagrams of a connector design for an electrode array, according to one embodiment of the present disclosure. The end of the LCP wiring strip contains a series of micro-contacts to connect with a ZA8 Ultra Low Profile Micro Array Compression Board from Samtec. These connector contacts remain flat (uncoiled) and stiffened to enable easy sliding into a series of alignment rails. Notches are added to ensure proper alignment of each lead. A cover with the ZA8 compression boards is placed on top of the connector contacts and pressed down by hand-twistable screws.

In some embodiments, methods of manufacturing ECoG and SEEG electrode arrays of the present disclosure include processes for fabricating leads with silicone and polyurethane, as described in FIG. 17, and for molding arrays in silicone. In some embodiments, electrode arrays using the processes and materials described herein were immersed in saline at an elevated temperature (e.g., 60° C.), resulting in a five-fold accelerated aging rate over body temperature (e.g., 37° C.). Exemplary results demonstrated that the electrode arrays of the present disclosure have an equivalent lifetime of about 5.5 years at 37° C., which complies with regulatory standards.

### 7. EXAMPLES

Example 1: As demonstrated in FIGS. 2 and 3, several generations of high-density, wired electrode arrays using LCP were developed. These devices enabled up to 25× higher spatial resolution sampling from the brain to improve epilepsy surgery outcomes. The LCP ECoG and SEEG electrode arrays have been thoroughly tested in chronic implants in animals (including non-human primates). Several different generations of these electrode arrays have also been tested intra-operatively in 10 humans. Results demonstrated the successful collection of high quality neural signals in all of these experiments, including propagating IIDs, HFOs and microseizures (FIGS. 5A-5D). Importantly, microseizures were observed occurring on just two electrodes spaced 750 μm apart, and were not visible on neighboring electrodes. Similarly, 88% of all detected HFOs occurred on a single channel, and were not detected on neighboring electrodes just 1.7 mm away (FIG. 7).

These observations underscore how the use of high-density ECoG and SEEG electrode arrays of the present disclosure are able to capture micro-scale electrographic phenomena that would be missed with conventional scale arrays.

As shown in FIG. 4, high-density SEEG electrodes were also fabricated. A wireless recording chip (FIG. 5) and external wireless relay station have been fabricated and tested as a part of the SEEG electrode array. In addition, two ASICs with 128 and 512 channel capacity per chip were integrated into the array (FIG. 5). Important technological features of both the ECoG and SEEG electrode arrays of the present disclosure include, but are not limited to, low cost manufacturing for high-density, biocompatible, thin-film devices at costs comparable to current ECoGs/SEEGs; near-hermetic circuit construction/packaging using thermally-bonded layers of liquid crystal polymer, rather than expensive titanium, glass or ceramic packages, as in traditional implantable devices; and fully-integrated, active system on chip (SOC) for signal acquisition from 1000 electrodes with self-testing and integrated amplification, multiplexing, stimulation and option for future wireless data and power.

In accordance with the above embodiments, active ECoG and SEEG electrode arrays were manufactured using a low-cost, (thin-film) manufacturing process with known biocompatible materials including LCP, gold (Au) and platinum iridium (Ptlr). Leveraging a low-cost, high volume manufacturing process allowed for the fabrication of active devices at a cost allowing approximately the same end user price ($400-$1,000) as current passive wired ECoG/SEEG. Other thin-film electrode arrays, such as those used in the Second Sight retinal implant, cost up to $10,000. As described herein, a process to fabricate wired, high-density surface (μECoG) and depth (μSEEG) electrode arrays using LCP has been demonstrated and validated.

Biocompatible metals (e.g., Au, PtIr and trace Pd) were used in the manufacture of the LCP-based electrode arrays of the present disclosure, although other biocompatible materials can also be used. In vitro tests demonstrated that LCP/Au thin film electrodes meet biocompatibility criteria guided by ISO 10993-5. Implantation of LCP electrodes in rabbit retina for 2.5 years showed no adverse effects.

Wiring of the ECoG and SEEG electrode arras of the present disclosure were composed of approximately 5 μm thick gold (Au) traces that can be as low as, for example, 20 μm wide and spaced 20 μm apart. Typical microelectrode trace resistance was measured as 2.7 Ω/cm. A 40 cm cable has trace resistance approximately 110Ω, an order of magnitude below the average microelectrode impedance of 2.8±0.8 kΩ at 1 kHz. Electrode contact yield (the number of functional microelectrode contacts per device) is approximately 99.5%. Wider traces were used for the macroelectrodes to improve yield and decrease trace resistance and required stimulator compliance voltage. All of the electrode contacts are plated with platinum iridium (PtIr) to reduce their impedance.

Currently approved neurological implantable devices have a system design that is largely unchanged from cardiac pacemakers. The electronics are enclosed in a titanium canister with ceramic or glass feedthroughs for connections to the electrodes. The number of connections from the electronics to the electrodes through the ceramic feedthroughs is not scalable beyond 16-64 connections, placing an upper limit on the spatial resolution of the arrays. Recently, two new technologies (Heraeus Cermat® and Schott Hermes®) allow high density and high resolution feedthroughs through ceramic or glass plates that are supposed to be hermetic. These plates are integrated into Ti cans or bonded to another ceramic to create a lidded package protecting the electronics. Although the ECoG and SEEG electrode arrays can include these technologies, processes have been developed to embed the electronics in a miniaturized LCP package that thermally-bonds to the LCP electrode substrate (FIG. 4). The thermal bonding process melts the two layers of LCP under heat and pressure, fusing them together into a single layer of LCP that cannot delaminate. The process creates a watertight seal around the electronics, which is important for enabling near-hermetic packaging for longer-term implants. Results described herein indicate that this process provides projected durability for at least 5 years. This packaging enables high-density feedthroughs for hundreds to thousands of electrodes, is low cost, and durable enough for the proposed 30 day clinical implantation (passive devices) and longer for research use.

Example 2: The electrode technology and active circuits described herein enable high-resolution sampling of micro-scale neural activity, far beyond the current standard of care (see, e.g., FIG. 6). Currently available implantable, wireless systems such as RNS and Medtronic RC+S have only four channels of wireless recording; the BRM Cereplex-I allows 128 channels but it is not hermetic. In contrast, the ECoG and SEEG electrode arrays of the present disclosure are configured to include up to 320 (passive) or 1024 (active) channels of recording and impedance testing, and multiple electrodes can be sampled simultaneously. This capability alternatively uses existing BRM 128 and 512 channel recording/stimulation ASICs, or a wireless IC, as described further herein. The wireless IC can be combined with the LCP-based ECoG and SEEG electrode arrays to create active μECoG and μSEEG electrode arrays. For example, as shown in FIG. 6, microscale recordings of epileptiform activity were obtained. Interictal events were intra-operatively recorded with a 244-channel LCP electrode from ten epilepsy patients before surgical resection. Microcontact recordings reveal a striking heterogeneity in the spatiotemporal dynamics of interictal events including spikes, microseizures and HFOs (FIG. 6).

Compared to SEEG electrode insertions, ECoG surgical procedures are highly invasive and can cause venous stasis and infarction inflammation, and other neuropathological changes. A 4-year retrospective review of clinical and patient-reported outcomes of 64 patients implanted with ECoG electrodes was performed. About 33% of patients had observable inflammatory changes beneath electrodes on neuropathological specimens; another 33% of patients had midline shift on post-implant neuroimaging; and nearly 50% the hospital days included the use of intravenous analgesics, indicating a moderate-severe level of breakthrough pain. These data reinforce the importance of improving patient experience and electrode tolerability and highlight the suitability of the proposed neuropathological, neuroradiological, and patient-report pain outcome measures.

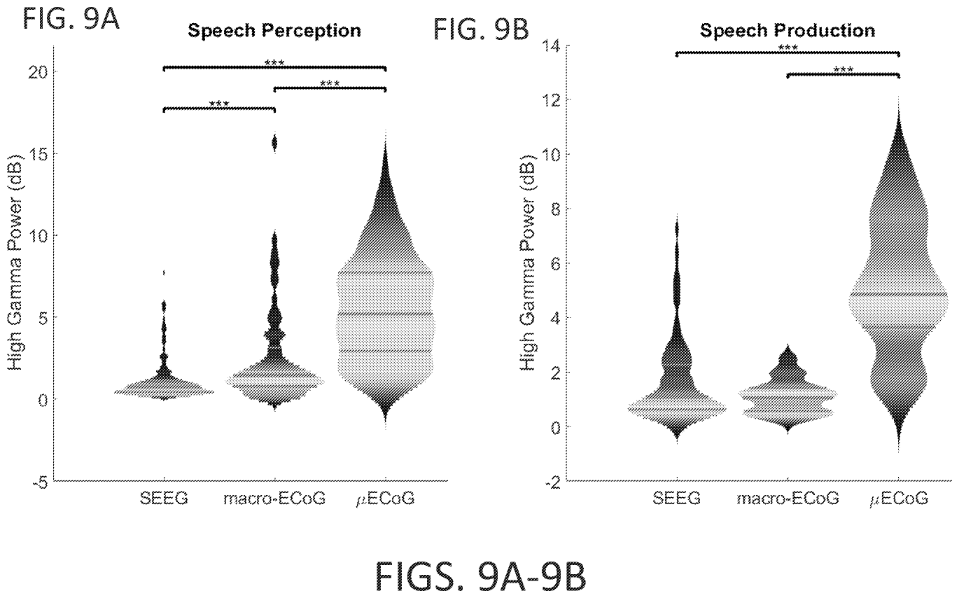

Example 3: Successful recordings were obtained from the μECoG arrays (FIG. 2) in human auditory and speech motor cortex during awake surgical procedures. The high-density, high-channel-count electrode arrays of the present disclosure produced much higher signal power in both auditory speech perception (FIG. 9A) and motor speech production (FIG. 9B) than conventional SEEG or macro-ECoG. Speech perception signals recorded with μECoG had an average evoked high-gamma (HG) power of 5.2 dB, but only 1.5 dB for macro-ECoG and 0.7 dB for SEEG. For motor speech, HG power averaged 4.8 dB for μECoG, 1.1 dB for macro-ECoG, and 1.0 dB for SEEG. These results demonstrate that the μECoG array captures neural activity with greater spatial resolution and SNR than SEEG or macro-ECoG.

Example 4: Results provided herein have determined that sub-millimeter electrode density can reliably predict HG μECoG potential fields in multiple species including human. Using spatial covariance analysis known as “kriging,” the real and theoretical interpolation error from electrode arrays recording sensorimotor areas at temporal frequencies up to 300 Hz were compared. To determine the ability to predict unsampled field potential, short windows of μECoG were interpolated and cross-validated from subsampled electrodes. The cross-validated interpolation error varied depending on statistical features of the spatial fields that were modeled as length-scale, texture, and SNR (FIG. 11). Kriging interpolation also yielded theoretical expected values for error. The cross-validated and model-based errors were compared over a large dataset of 500 ms-windowed μECoG, and the results demonstrated that expected values explained interpolation residuals with high accuracy in recordings from rat, monkey, and human (r\hat{ }{2}>0.98).

Electrode spacing term was manipulated in covariance models to normalize the expected interpolation error to 10% of the process variance in each short-time window. The resulting distribution of electrode spacings was termed the kriging resolution. The 5^{th} percentile of kriging resolutions was established as the electrode spacing that would be effective for <10% interpolation error in 95% of cases. The required spacing varied across species, and across temporal bandpass. The spacing used to interpolate HG (75-150 Hz) μECoG in human auditory cortex under anesthesia was 663 μm, with the assumption of noise-free conditions.

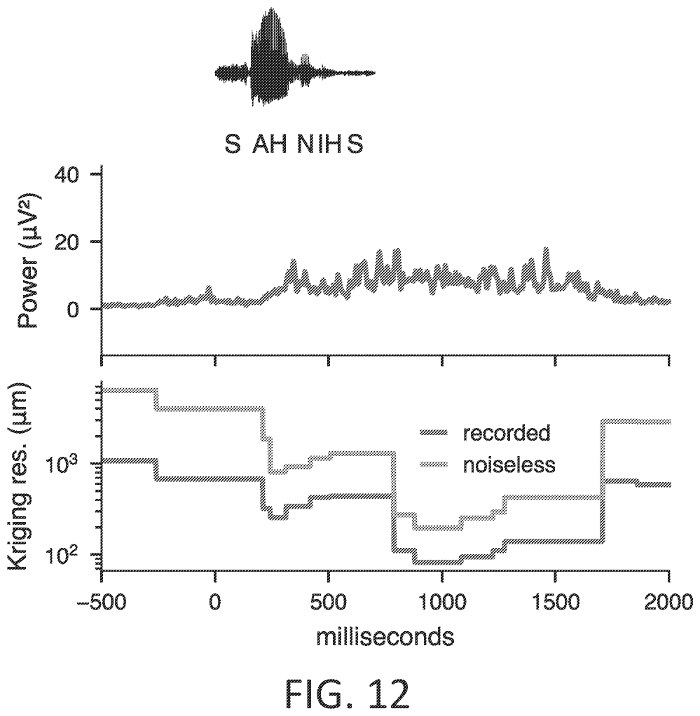

Example 5: The spatial resolution used for optimal sampling of neural signals in the human brain was determined. During a speech trial, the electrode spacing used to predict HG μECoG signals dropped to 200 μm for noise-free conditions and <100 μm when including recording noise (FIG. 12). A provisional log-linear regression of kriging resolution was made for HG power modulation in 200-2000 ms post-stimulus windows of 332 trials. The fit was significant (F_{1,3273}=1232, p<<0.001) and the model demonstrated that kriging resolution spacing reduced by a factor of 0.76 for every dB increase in HG response, ranging from 2.90 (2.78-3.04) mm at −3 dB to 0.55 (0.51-0.58) mm at 6 dB (mean and 95% confidence interval).

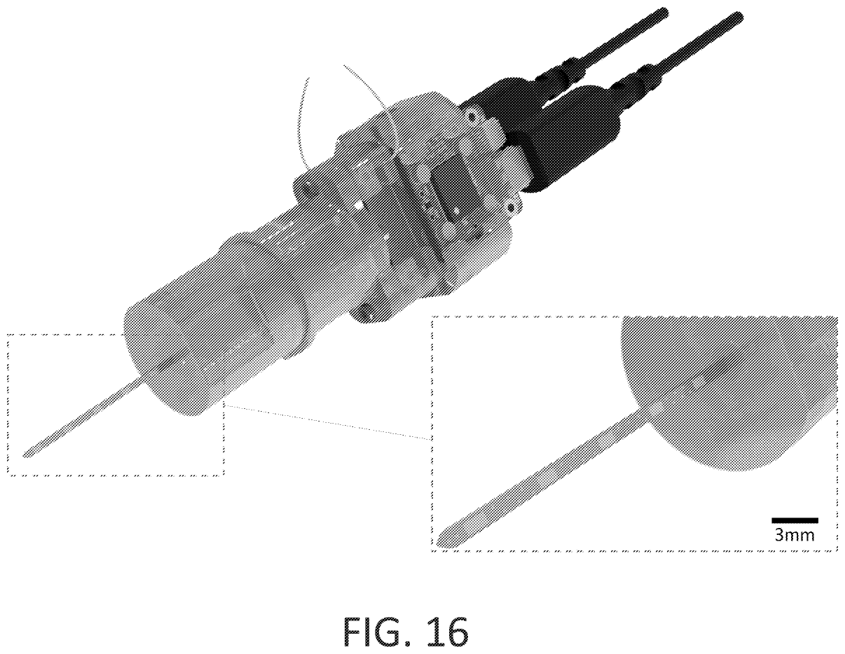

Wireless arrays that are successfully tested in vitro will be implanted in nonhuman primates (NHPs) for at least one month using a recording chamber system designed for wireless μSEEG and μECoG (FIG. 16). Experiments will be performed to measure the resolution of the electrode arrays and their long-term reliability. Optogenetic stimulation is a technique that uses light to stimulate neural activity and has been recently published in NHP. Recordings will be obtained from the wireless μECoG and μSEEG devices of the present disclosure as optogenetic stimulation is delivered. Unlike electrical stimulation, which activates neuronal cells and axons unpredictably, optogenetic stimulation can be used to create a defined source of neural signals in the brain. Analyzing the response to stimulation will also allow for the quantification of tissue excitability. To assess long-term signal reliability, recordings will be obtained using wireless μECoG and μSEEG devices of the present disclosure, as monkeys perform behavioral tasks in which they earn rewards by reaching to press buttons in different locations.

Example 6: Experiments will be conducted to obtain reliable, high-resolution recordings from wireless μECoG and μSEEG arrays in NHPs. Using the semi-chronic recording chamber system will allow for the rapid iteration of device designs as needed, without requiring additional animals or surgeries. Wired LCP electrode designs were successfully tested in NHPs. The semi-chronic chamber system has enabled implanting and the successful testing of even very fragile electrodes with limited ability to bend sharp corners. The LCP electrodes of the present disclosure will be much more durable.

Ultimately, to demonstrate the ability of the wireless electrode arrays of the present disclosure to capture clinically-relevant electrographic signals, intra-operative recordings will be performed in epilepsy patients undergoing respective surgery. Recordings will be obtained from the cortex intraoperatively from patients undergoing surgical evaluation for pharmacologically-resistant epilepsy. The wireless μECoG array will be placed on the surface of the brain. The wireless module portion of the arrays will not come in contact with the patient. The electronics will be supported by a holder above the head to prevent any risk to the patient in the unlikely event of package failure. The wireless arrays allow for recording with up to 320 electrodes in up to 10 patients to demonstrate high-quality recordings of neural signals, with comparable signal characteristics to wired LCP μECoG arrays.

The auditory response sensitivity of intraoperative μECoG recording will be compared to those of in-unit recordings with macro-ECoG and SEEG. The strength and reliability of the HG response are critical for speech decoding algorithms. For all recordings, the HG power modulation ratio over baseline will be measured during speech perception and production. A 3.9 dB mean difference was observed in modulation ratio in preliminary results (FIG. 9). Taking that gain as representative of a typical implant, about 10 subjects would be needed to detect a 3 dB doubling of mean modulation power for μECoG versus macro-ECoG or SEEG with a statistical power of 0.9, using a paired sample t-test (one-sided, p<0.05) and a conservative standard deviation of 3 dB. Forty subjects would bring the power to 0.997.

### 8. MATERIALS AND METHODS

Use in Human Subjects. Clinical investigations can be designed to test (1) the safety and tolerability of implanting thin film (TF) electrode arrays of the present disclosure, and (2) the efficacy and reliability of the signals measured with TF electrode arrays. As described further herein, the TF electrode arrays are configured similarly to current technology and will provide comparable recordings with improved safety and tolerability. Studies can be conducted to demonstrate that TF electrode arrays of the present disclosure yield ECoG and SEEG recordings and with similar quality as CG electrodes and to assess secondary outcomes that will inform the design and demonstrate the superior safety and tolerability of TF electrodes over CG electrodes. As described further herein, the enhanced spatial resolution and improved signal-to-noise ratios for high frequency activity enabled by the addition of microcontacts to the ECoG and SEEG arrays of the present disclosure has added utility to delineating the epileptogenic zone compared to conventional electrodes.

Inclusion criteria for patient cohorts can include: (1) drug-resistant focal epilepsy with suspected neocortical origin as identified through review of seizure history, seizure semiology, presurgical EEG, neuroimaging, and neuropsychological data at multidisciplinary epilepsy surgery conference (2) multidisciplinary conference recommendation for subdural grid implantation; and (3) fluency in English or Spanish. Exclusion criteria for patients will be: (1) a neurological illness or serious medical problems (other than epilepsy); (2) estimated IQ within the intellectual disability range as assessed during pre-operative neuropsychological testing; (3) current substance abuse; (4) active severe psychiatric symptoms; (5) previous neurosurgery in the targeted region and (6) surgical treatment that includes resection of brain tissue prior to grid implantation. Results are intended to identify critical device design features and outcomes that will guide the development of a subsequent large-scale clinical trial for epilepsy treatment.

The various exemplary outcome measures described below can be used to assess the ECoG and SEEG electrode arrays of the present disclosure in clinical studies.

Primary outcome measures include:

Serious Adverse Event (SAE) Frequency: Frequency of SAEs in the perioperative period (post-operative day 0 to 3 months) in TF compared to clinical grid (CG) implanted patients. SAEs of interest include: intracranial hemorrhage, venous or arterial infarct, infection, seizure cluster or status epilepticus requiring use of intravenous medications, and neurologic deterioration related to cerebral edema.

Signal to noise ratio (SNR): Electrode SNR will be calculated as follows: computed spectral density over a 10 minute epoch of awake, interictal ECoG selected blindly by the EEG technologist on post-implant day 1 and final day of implant. Spectral density will be estimated by averaging spectral estimates from non-overlapping 1 second recording segments employing multitaper methods with 4 Hz frequency smoothing. The SNR will be assessed for conventional ECoG signals as the as the ratio of the integrated power from 5-50 Hz divided by the power at 60 Hz. Spectral power estimates will be transformed with a logarithm to stabilize the variance before averaging. SNR metrics can be computed in each of the 64 contacts of the CG and macro-contacts of the TF electrodes and determine the change in SNR from post-operative day 1 and right before explant. In addition, the intra-cluster correlation in SNR between contacts can be calculated within a “bank” of 16 contacts and the entire grid for each subject in the CG and TF groups.

Midline shift: Degree of midline shift in CG and TF implanted patients as measured on post-operative day 1 CT. A board-certified neuroradiologist will review the 3-D volumetric 1 mm slice thickness CT scans obtained on post-operative day 1 as part of routine clinical care in reformatted all 3 planes relative to the inter-commissural plane and measure the degree of midline shift (in mm) at the level of the foramen of Monroe.

Patient-reported pain scores: Daily post-operative patient reported pain scores using a visual analog scale (VAS) and Brief Pain Inventory in CG implanted patients from post-operative day 1 until the time of electrode explant. VAS and BPI scores can be analyzed by estimating adjusted group mean at each time point while controlling for baseline test performance and other factors known to influence performance on patient-reported pain measures, specifically baseline mood during routine pre-surgical neuropsychological evaluation

Seizure timing and onset zone reliability: Compare level of agreement between expert clinical neurophysiologist defining the time of seizure onset and the seizure-onset zone in CG- and TF electrode implanted patients using macrocontacts only. Three blinded experts will review intracranial EEG recorded from CG controls or the macrocontacts of TF-implanted subjects to determine the time of first electrographic change and the contacts involved in the first 5 seconds of the ictal discharge. Intraclass-correlation coefficient can be compared for time of seizure onset obtained for control and experimental group for timing of seizure onset. To assess the reproducibility of defining the distribution of contacts involved in the seizure-onset, the degree of agreement can be defined as the mean Spearman Rho of the spatial correlation between the three rater pairs (Rater 1 vs Rater 2, Rater 1 vs Rater 3 and Rater 2 vs Rater 3).

Stimulation mapping parameters: In patients who undergo DCS mapping to identify eloquent cortex as part of routine clinical care, compare mean charge density (μC/cm²) required to elicit a clinical response in language, motor and sensory cortices (evaluated separately) in TF- and CG electrode patients.

Qualitative tissue neuropathology assessment: In patients undergoing resection of epileptogenic tissue, a blinded board-certified neuropathologist will review neuropathology slides obtained as part of routine tissue assessment of epilepsy specimens (adjacent sections stained with Luxol fast blue, hematoxylin and eosin, and immunostaining for GFAP, CD34, CD56, synaptophysin, NeuN immunostain, and Ki-67 immunostain) and grade the degree of acute inflammatory changes. Inflammation severity will be graded based on microglial activation/proliferation, inflammatory cell infiltrate and tissue damage: 0—absent, 1—very mild, 2—mild, 3—moderate, 4—moderate-severe and 5—severe.

Qualitative neurophysiological assessment: In addition to SNR, other factors may influence the quality of the recorded ECoG signal such as susceptibility to movement-related artifacts that more difficult to quantify. ECoG signals will also be graded on a qualitative scale (A grade of 0 is for near perfect, for records without electrode artifact/obscuration, and the severity of degraded recordings will be graded: 1—minimal, 2—mild, 3—moderate and 4—severe). Five minute signals will be selected by a blinded EEG technologist during wakefulness at day 1 and the final day of recordings.

Seizure timing and onset zone definition based on TF electrode microcontacts: In patients implanted with the TF array, examine the spatial extent (in cm² of cortex) of the seizure onset zone as defined by activity at microcontacts versus macrocontacts on the same array. One microcontact will be selected at random from the four microcontacts nearest each macrocontact to created matched, numbered sets of micro- and macrocontacts with similar spatial layout. Seizure onset zone will be defined for microcontacts using both standard clinical low-frequency signal and high-frequency bands. Three blinded clinical electrophysiologists raters will perform the assessments and inter- and intra-rater reliability will be compared.

Seizure outcomes: Examine seizure outcomes at 1 year using Engel score in TF electrode implanted patients and compare results in patients where the microgrid-defined seizure onset zone was within the resection margin and when it extended beyond the margin.

Source of materials. Study materials generally include biospecimens (brain tissue) and outcome measurement data obtained from analysis of biospecimens, as well as clinical data abstracted from medical records, intracranial EEG data recorded during the hospital stay in the EMU or electrocorticograms recorded at the time of surgery, brain MRI images, pain survey scores, and neuropsychology assessment scores. All study data will be collected by the investigators and/or other study personnel including Blinded Reviewers. Data collected for the clinical trial will be entered directly into electronic case report forms (eCRF) developed by DataCore programmers into the TrialMaster® electronic data capture (EDC) system. TrialMaster is a web-based software solution that allows clinical trial sponsors and investigative sites to collect data easily and securely, validate, transmit and analyze clinical study data including patient histories, adverse events and other clinical trial related information. The TrialMaster EDC system has been designed and developed to be in compliance with 21 CFR Part 11, electronics records, electronic signatures and predicate rules when implemented and controlled effectively. Specifically, the 21 CFR part 11 features are: user level management (role-based security controls), file integrity (versioning and tamper resistance), audit trails (date/time stamp for all record changes), and electronic signatures (via username/password). TrialMaster also incorporates and eLearning module to train all users in the use of the system. The eCRF design will emphasize a clean, uncluttered layout, with features incorporating visual cues for the clinical center staff recording the data, such as logical data groupings, drop-down lists, checkboxes or free text as appropriate, dynamic forms that incorporate skip logic, and field edit checks (ranges and rules) that automatically validate the data being entered. Ongoing data management support will consist of manual CRF data reviews, query, resolution, external data reconciliation, automated adverse event coding, and other tasks as appropriate. At the conclusion of the clinical trial, data management will perform data cleaning, query resolution and required reconciliation as specified in the data management plan. After database lock, a full data extract will be provided to biostatistics for analysis, transformation and loading into an archival repository.

To protect participants' confidentiality, participants will be identified by a unique alpha-numeric code (see below); all data from that participant will be stored using this code. Histopathology tissue slides biospecimens will labeled using a barcode that links to the alpha-numeric code. A subject identification list will contain the mapping between the alpha-numeric code and the identity of each participant. Intracranial EEG will be deidentified and converted to EDF format using tools native to the clinical EEG software (e.g., Natus Neuroworks). An additional review of the text component of the EDF file will be performed by study coordinator to ensure no PHI elements are included as comments or annotations.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof

What is claimed is:

1. An electroencephalography (EEG) device comprising:
an electrode array comprising a plurality of microelectrodes and macroelectrodes uniformly or non-uniformly arranged in a polymer-based substrate; and
a signal acquisition component coupled to the electrode array configured to collect and transmit electrical signals obtained from a subject's brain.

2. The device according to claim 1, wherein the electrode array is configured to perform electrocorticography (ECOG) on the subject.

3. The device according to claim 1, wherein the electrode array is configured to perform stereoelectroencephalography (SEEG) on the subject.

4. The device according to any of claims 1 to 3, wherein at least a portion of the electrode array or the signal acquisition component comprise silicone molding.

5. The device according to any of claims 1 to 4, wherein at least a portion of the electrode array is coated in silicone having a thickness from 0.2 mm to 2.0 mm.

6. The device according to any of claims 1 to 5, wherein the polymer-based substrate comprises a liquid crystal polymer (LCP).

7. The device according to any of claims 1 to 6, wherein the device further comprises a circuit coupled to the electrode array and configured to amplify and/or digitize the electrical signals.

8. The device according to any of claims 1 to 7, wherein the signal acquisition component is coupled to a clinical data acquisition system.

9. The device according to any of claims 1 to 8, wherein the signal acquisition component comprises at least one coiled lead comprising a plurality of microwires extending from contacts points on the plurality of microelectrodes and macroelectrodes.

10. The device according to claim 9, wherein the at least one coiled lead comprises a single lead comprising the plurality of macroelectrodes.

11. The device according to claim 9, wherein the at least one coiled lead comprises from 2 to 20 leads comprising the plurality of microelectrodes.

12. The device according to claim 9, wherein the at least one coiled lead comprises from 10 to 500 channels per lead.

13. The device according to claim 9, wherein the at least one coiled lead comprises a diameter of at least 1.0 mm.

14. The device according to any of claims 1 to 8, wherein the signal acquisition component comprises a wireless chip.

15. The device according to claim 14, wherein the wireless chip is contained within a polymer-based substrate.

16. The device according to claim 14, wherein the wireless chip is configured for implantation in the subject.

17. The device according to claim 14, wherein the wireless chip is a 65,000 electrode wireless recording chip.

18. The device according to any of claims 1 to 17, wherein the electrode array comprises from 4 to 500 uniformly or non-uniformly arranged macroelectrodes.

19. The device according to any of claims 1 to 18, wherein the macroelectrodes comprise diameters from 1 mm to 10 mm.

20. The device according to any of claims 1 to 19, wherein the macroelectrodes are spaced from 0.2 cm to 5 cm apart from each other.

21. The device according to any of claims 1 to 20, wherein the electrode array comprises from 100 to 10,000 uniformly or non-uniformly arranged microelectrodes.

22. The device according to any of claims 1 to 21, wherein the microelectrodes comprise diameters from 1 μm to 1 mm.

23. The device according to any of claims 1 to 22, wherein the microelectrodes are spaced from 100 μm to 5 mm apart from each other.

24. The device according to any of claims 1 to 23, wherein the electrode array provides at least a 2-fold increase in spatial sampling resolution compared to an array comprising only macroelectrodes.

25. An electrocorticography (ECOG)-stereoelectroencephalography (SEEG) combination device comprising:
an ECOG component comprising an electrode array comprising a plurality of microelectrodes and macroelectrodes uniformly or non-uniformly arranged in a polymer-based substrate, and a signal acquisition component coupled to the electrode array configured to collect and transmit electrical signals obtained from a subject's brain; and
an SEEG component comprising an electrode array comprising a plurality of microelectrodes and macroelectrodes uniformly or non-uniformly arranged in a polymer-based substrate, and a signal acquisition component coupled to the electrode array configured to collect and transmit electrical signals obtained from a subject's brain.

26. A method of manufacturing any of the EEG devices of claims 1 to 25, the method comprising:
arranging the plurality of microelectrodes and macroelectrodes uniformly or non-uniformly within a polymer-based substrate to form the electrode array; and
coating at least a portion of the array and the signal acquisition component with a composition comprising silicone.

27. An electroencephalography (EEG) system comprising:
an electrode array comprising a plurality of microelectrodes and macroelectrodes uniformly or non-uniformly arranged in a polymer-based substrate;
a signal acquisition component coupled to the electrode array configured to collect and transmit electrical signals obtained from a subject's brain; and
a clinical data acquisition system.

28. The system according to claim 27, wherein the electrode array is configured to perform electrocorticography (ECOG) on the subject.

29. The system according to claim 27, wherein the electrode array is configured to perform stereoelectroencephalography (SEEG) on the subject.

30. The system according to any of claims 27 to 29, wherein the system comprises an electrode array configured to perform electrocorticography (ECOG) on the subject, and an electrode array configured to perform stereoelectroencephalography (SEEG) on the subject.

31. The system according to any of claims 27 to 30, wherein the device further comprises a circuit coupled to the electrode array and configured to amplify and/or digitize the electrical signals.

32. The system according to any of claims 27 to 31, wherein the signal acquisition component comprises at least one coiled lead comprising a plurality of microwires extending from contacts points on the plurality of microelectrodes and macroelectrodes.

33. The system according to any of claims 27 to 32, wherein the signal acquisition component comprises a wireless chip.

34. A method of evaluating a subject for a neurological impairment, the method comprising:
recording electrical signals in a portion of a subject's brain using the any of the devices of claims 1 to 25; and
evaluating the subject based on the recorded electrical signals.

35. The method according to claim 34, wherein neurological impairment comprises a seizure.

36. The method according to claim 34 or claim 35, wherein evaluating the subject comprises determining the presence or absence of the neurological impairment, and/or determining the severity of the neurological impairment.

37. The method according to any of claims 34 to 36, wherein evaluating the subject comprises identifying means for treating the neurological impairment.

38. The method according to any of claims 34 to 37, wherein the method further comprises treating the neurological impairment in the subject, wherein the treatment comprises pharmacological intervention, surgical intervention, and/or electrophysiological intervention.

39. A method for treating a subject having or suspected of having a neurological impairment, the method comprising:
recording electrical signals in a portion of the subject's brain using an electrode array comprising: a plurality of microelectrodes and macroelectrodes uniformly or non-uniformly arranged in a polymer-based substrate, and a signal acquisition component comprising a wireless chip coupled to the electrode array configured to collect and transmit electrical signals obtained from a subject's brain; and
administering an electrical pulse to at least a portion of the subject's brain to treat the neurological impairment.
